(19) United States
(12) Patent Application Publication
(10) Pub. No.: US 2011/0185440 A1
(43) Pub. Date: Jul. 28, 2011

(54) CISD2-Knockout Mice and Uses Thereof

(75) Inventors: Ting-Fen Tsai, Taipei (TW); Yi-Fan Chen, Taipei (TW); Shih-Feng Tsai, Miaoli County (TW); Ya-Ting Chen, Miaoli County (TW)

(73) Assignees: NATIONAL YANG-MING UNIVERSITY, Taipei City (TW); NATIONAL HEALTH RESEARCH INSTITUTES, MIAOLI COUNTY (TW)

(21) Appl. No.: 13/041,821

(22) Filed: Mar. 7, 2011

Related U.S. Application Data
(63) Continuation-in-part of application No. 12/481,042, filed on Jun. 9, 2009, now abandoned.
Application No. 12/481,042 is a continuation-in-part of application No. 11/866,374, filed on Oct. 2, 2007, now abandoned.
(60) Provisional application No. 60/849,089, filed on Oct. 3, 2006.

(51) Int. Cl. A01K 67/027

(52) U.S. Cl. 800/18

(57) ABSTRACT

The present invention is related to a Cisd2 knockout mouse with phenotype comprising mitochondrial breakdown and dysfunction, wherein Cisd2 is defined as SEQ ID NO. 1. The present invention is also related to a mouse model of Wolfram Syndrome 2 (WFS2) disease consisting of a Cisd2 knockout mouse. The present invention is further related to a method for screening a candidate agent for preventing or treating WFS2 disease.

### CROSS-REFERENCE TO RELATED APPLICATIONS

This application is a Continuation-in-part of the pending U.S. patent application Ser. No. 12/481,042 filed on Jun. 9, 2009, which is a Continuation-in-part U.S. patent application Ser. No. 11/866,374 of which claims priority U.S. Application No. 60/849,089 filed on Oct. 3, 2006, all of which is hereby incorporated by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the entire prior art of record and any search that the Office deems appropriate.

### FIELD OF THE INVENTION

The present invention relates to a premature aging or Wolfram syndrome 2 (WFS2) animal model and use thereof.

### BACKGROUND OF THE INVENTION

Aging, or organismal senescence, is defined as gradual changes in an organism that “adversely affect its vitality and function, but most importantly, increases the mortality rate of an organism as a function of time”.

Aging can be characterized as the age-related decline of physiological functions necessary for the survival and reproduction of an organism. Common age-associated diseases connected to these functions include, but not limited to, arteriosclerosis, cancer, dementia and osteoporosis. To understand the primary causes of these diseases' onset and commencing of generalized malfunctions of multiple organ systems that potentially shorten life span and reduce fertility is central to understanding human aging.

A number of genetic components of aging have been identified using model organisms, ranging from the bakers' yeast (Saccharomyces cerevisiae), the soil roundworm (Caenorhabditis elegans), the fruit fly (Drosophila melanogaster), and the mouse (Mus musculus).

One approach to understanding the molecular basis of human aging is to find genes that determine inherited premature aging syndromes thereby causing rapid development of these senescence associated diseases early in life. To that end, mutant mice that display multiple phenotypes resembling accelerated aging have been developed in recent years. However, virtually all of them display partial spectrum of the senescence associated phenotypes.

CISD2 is the second member of the gene family containing the CDGSH iron sulfur domain. There are currently three members in this gene family: CISD1 (synonym ZCD1, mitoNEET), CISD2 (synonym ZCD2, Noxp70, Miner1) and CISD3 (synonym Miner2). CISD1 is an outer mitochondrial membrane protein that was originally identified as a target protein of the insulin sensitizer drug pioglitazone used to treat type 2 diabetes. CISD1 protein contains a transmembrane domain, a CDGSH domain and a conserved amino acid sequence for iron binding; biochemical experiments suggest that CISD1 is involved in the control of respiratory rates and regulates oxidative capacity. However, CISD2 and CISD3 are novel genes with previously uncharacterized functions. The only molecular documentation for CISD2 is that CISD2 was one of the markers for early neuronal differentiation in a cell culture study.

Recently CISD2 gene has been identified as the second causative gene associated with Wolfram syndrome (WFS; MIM 222300), which is an autosomal recessive neurodegenerative disorder. Wolfram syndrome is highly variable in its clinical manifestations, which include diabetes insipidus, diabetes mellitus, optic atrophy and deafness; thus, it is also known as the DIDMOAD syndrome. Positional cloning and mutation studies have revealed that WFS is a genetically heterogeneous disease with a complex molecular basis involving more than one causative gene in humans. A portion of WFS patients belonging to the Wolfram syndrome 1 group (WFS1; MIM 606201) carried loss-of-function mutations in the WFS1 (wolframin) gene, which encodes a transmembrane protein primarily localized in the endoplasmic reticulum (ER). In addition to this, a homozygous mutation of CISD2 gene has been identified in three consanguineous families with Wolfram syndrome and these patients have been classified as Wolfram Syndrome 2 (WFS2; MIM 604928). However, the function of the CISD2 protein in these patients and in all other organisms remains unknown and its physiological role has not been explored.

Significantly, CISD2 gene is located within the region on human chromosome 4q where a genetic component for human longevity has been mapped. Previously a research studied 137 sets of extremely old siblings (308 individuals in all) and conducted a genome-wide scan search for predisposing loci that might confer longevity; this linkage study revealed a single region on chromosome 4q and suggests that there may be at least one master gene contributing to lifespan control; however, the responsible gene has not been identified.

### SUMMARY OF THE INVENTION

The present invention provides a Cisd2 knockout mouse with phenotype comprising mitochondrial breakdown and dysfunction, wherein Cisd2 is defined as SEQ ID NO. 1.

The present invention also provides a mouse model of Wolfram Syndrome 2 (WFS2) disease consisting of a Cisd2 knockout mouse.

The present invention further provides a method for screening a candidate agent for preventing or treating WFS2 disease comprising: (a) providing the mouse of claim 1; (b) adding said candidate agent into said mouse, and (c) determining the agent by identifying the desired therapeutic effects in ameliorating WFS2 disease associated phenotypes.

### BRIEF DESCRIPTION OF THE DRAWINGS

To adequately describe the present invention, references to embodiments thereof are illustrated in the appended drawings. These drawings herewith form a part of the specification. However, the appended drawings are not to be considered limiting in their scope.

FIG. 1 shows genes and markers located in the human chromosome 4q23-4q25 region, to which the human longevity locus was previously linked and was deemed to be near marker D4S1564 (1A). Cisd2, the human ortholog of the mouse gene targeted in the present invention, is located between the ubiquitin-conjugating enzyme E2D 3 (UBE2D3) and 3-hydroxybutyrate dehydrogenase type 2 (BDH2) genes illustrated in FIG. 1A. (1B) Northern blot analysis of Cisd2 mRNA expression in mouse tissues. A corresponding 18S rRNA band for each lane is used to assist in normalizing the band intensity on the Northern blot. (1C) Quantitative real-time RT-PCR of Cisd2 mRNA using brain RNA isolated from 2-, 3-, 6-, 15- and 28-month old wild-type C57BL/6 mice. Significant decrease of the Cisd2 mRNA was detected in the naturally aged mice, i.e. 15- and 28-month old mice, indicating that expression levels of Cisd2 decrease in an age-dependent manner.

FIG. 2 shows the genomic structure of the wild-type and the resulting targeted alleles of the Cisd2 gene. (2A) The Cisd2 gene was disrupted by a targeted insertion vector containing puromycin (Puro) selection cassette. A probe used to identify the targeted events by Southern blot analysis is indicated along with the diagnostic EcoRI sites in the Cisd2 gene and the replacement region from the targeting construct. (2B) Southern blot hybridization of tail DNA isolated from wild-type (+/+), heterozygous (+/−) and homozygous (−/−) offspring of a heterozygous intercross using a 3′ flanking probe. (2C) Northern blot analysis of Cisd2 mRNA isolated from brain tissues of 8-week old wild-type (+/+), heterozygous (+/−) and homozygous (−/−) offspring. (2D) Illustrates pedigree of three generations of mice carrying the Cisd2 mutant allele, with mice grouped by sex and genotype. Square, male; circle, female; checkered symbol, chimera; open symbols, Cisd2 wild-type; filled symbols, Cisd2 homozygous (−/−) knockout; half filled symbols, Cisd2 heterozygous (+/−) knockout.

FIG. 3 shows summary of the aging-related phenotypes as a function of age in the Cisd2⁻⁻ mice. (3A) The timing of the onset of each phenotype approximates the average age of onset for that phenotype; wk, week. The onset age for each mouse for each phenotype shows variation around the average onset age to a limited degree. (3B) Decreased survival rate of the Cisd2^{-/-} mice. The percent survival of wild-type (+/+, n=49), heterozygous (+/−, n=22) and homozygous (−/−, n=16) mice including males and females is plotted against the age in months. (3C) Growth curve of male and female mice with different genotypes. Body weight is plotted against age of mice in weeks.

FIG. 4 shows premature aging related symptoms, including hair graying, protruding ears, and prominent eyes, in 48-week-old Cisd2^{-/-} mice (4A). (4B) 24-week-old Cisd2^{-/-} mice develop blindness. (4C) Opacity of cornea analyzed by histological examination. The H&E stain indicated collagen deposition in the lesion outside the cornea in Cisd2^{-/-} mice. (4D) Early depigmentation of the fur in 48-week-old Cisd2^{-/-} mice. (4E) Hair follicle atrophy visualized by Masson's trichrome staining in 48-week-old Cisd2^{-/-} mice. (4F) Reduced percentage of hair follicle with hair in 48-week-old Cisd2^{-/-} mice relative to that for age-matched heterozygous mice (+/−) and wild-type (+/+). (4G) The opacity of cornea was analyzed by histological examination. (4H) Representative photographs of 48-week old Cisd2^{-/-} and age-matched heterozygous Cisd2^{+/-} female mice 13 days after removal of hair from a 2-cm2 dorsal area. (4I) Quantification of hair re-growth for the Cisd2^{-/-} and Cisd2^{+/-} female mice. For the hair re-growth analysis, mice were shaved on the dorsal surface with a razor under anesthesia. Hair re-growth was measured 20 days after shaving as described previously.

FIG. 5 shows cross sections of skin from 48-week-old Cisd2^{+/+} and age-matched Cisd2^{-/-} mice, respectively (5A and 5B). (5C) Quantification of the subcutaneous muscle tissue, adipose tissue and dermis for the histological sections of the wild-type and Cisd2^{-/-} skins. *p<0.05 was considered statistically significant.

FIG. 6 shows micro-computed tomography imaging of the trabeculae in the femur of 4-month-old wild-type (+/+) and age-matched homozygous (−/−) mice (6A). (6B) Femur density of wild-type (+/+), heterozygotes (+/−), and homozygotes (−/−), was analyzed by dual energy x-ray absorpitometer (DEXA). (6C) Whole-body radiography of a 4-month-old wild-type (+/+) and homozygous (−/−) mouse. (6D) Micro-computed tomography scanning for 3D reconstruction of thoracic and spinal columns of a 5-month-old wild-type (+/+) and an age-matched homozygous (−/−) mouse. (6E) A decrease of mean thoracic volume in a homozygous (−/−) versus an age-matched wild-type (+/+) mouse. (6F and 6G) Comparing 2 respiratory parameters, mean tidal volume and enhanced pause, respectively, between various age-matched homozygous (−/−) vs. wild-type (+/+) mice, *p<0.05; **p<0.005. (6H) Plethysmographs of wild-type and Cisd2^{-/-} mice, respectively. (6I and 6J) H&E staining of transverse sections of skeletal muscle of 4-week old and 28-month old wild-type mice. (6K and 6L) Muscle degeneration of 4- and 8-week old Cisd2^{-/-} mice which was examined by H&E staining of transverse sections of the skeletal muscle. Black arrows indicate degenerated transverse fibers that are present in the Cisd2^{-/-} and also in spontaneously aged mice. The blue arrow indicates an angular fibre, which is an indicator of muscle atrophy caused by neuron degeneration. (6M) Quantification of the degenerating fibers in the skeletal muscles. *p<0.05; **p<0.005.

FIG. 7 shows an electron micrograph of section of muscle from a wild-type (+/+) mouse (7A). (7B) A similarly prepared section of muscle from a homozygous (−/−) mouse. (7C) An electron micrograph of the degenerated margin of striated muscle cell. (7D) The degenerated margin of injury striated muscle cell. The debris (D) of muscle cell and degenerated myofilaments (arrows) were separated from muscle cell. Myf, myofibril; N, nucleus; M, mitochondrion; V, digestive vacuole; Ly, lysosome.

FIG. 8 shows a transversely sectioned myelinated nerve fiber from the peripheral nerves of skeletal muscle of wild-type and Cisd2^{-/-} mice, respectively. (8A and 8B) The axon is enveloped by the myelin sheath (MS) formed by fusing many layers of Schwann cell plasma membrane. Myelin sheath degeneration, highlighted by asterisks, was detected only in the Cisd2^{-/-} mice. (8C) RT-PCR analysis of BDNF, NT-3 and TrkB mRNA isolated from brain of 3-month old wild-type and Cisd2 homozygous mice. Hprt and Actb are used as internal controls. BDNF, brain-derived neurotrophin factor; NT, neurotrophin, Trk, tyrosine receptor kinase; Hprt, Hypoxanthine guanine phosphoribosyl transferase; Actb, beta-actin. (8D) Relative quantification by real-time PCR of BDNF mRNA isolated from the brain of various ages of wild-type mice (gray bars) and different genotypes of the Cisd2 knockout mice (black bars). (8E) A myelinated axon of sciatic nerve from a Cisd2^{-/-} mouse. An ovoid with a disintegrating myelin sheath and a degenerating axonal component are shown. (8F) Debris from an axon undergoing degeneration in the Cisd2^{-/-} sciatic nerve.

FIG. 9 shows wild-type mitochondria in the brain (hippocampus) (9A). (9B) A Cisd2^{-/-} mitochondrion in the brain (hippocampus). Note the outer mitochondrial membrane has broken down (arrow head) while the inner cristae appear to be intact. (9C) Cisd2^{-/-} mitochondria in sciatic nerve. One mitochondrion (arrow head) has a destroyed outer membrane, but with cristae still visible; the other mitochondrion (arrow) has destroyed outer and inner membranes. (9D) Wild-type mitochondria in cardiac muscle. (9E) Cisd2^{-/-} mitochondria in cardiac muscle. This micrograph shows one mitochondrion (arrow head) with a destroyed outer membrane and two degenerated mitochondria consisting of debris (arrows). (9F) A cluster of autophagic vacuoles and abnormal mitochondria which was observed between the myofibrils of Cisd2^{-/-} skeletal muscle (white arrows). (9G) A wild-type myelinated axon of the sciatic nerve. N, nucleus of Schwann cell; MS, myelin sheath. (9H) A myelinated axon of sciatic nerve from a Cisd2^{-/-} mouse. An ovoid with a disintegrating myelin sheath and a degenerating axonal component are shown. (9I) Debris from an axon undergoing degeneration in the Cisd2^{-/-} sciatic nerve.

FIG. 10 shows early occurrence of mitochondrial destruction, myelin sheath disintegration and axonal lesions in 2-week old Cisd2^{-/-} mice. (10A) A cluster of degenerating mitochondria, autophagic vacuoles and debris is generated between myofibrils (arrows) of Cisd2^{-/-} cardiac muscle. (10B) A late or degradative autophagic vacuole (AVd) enclosing a mitochondrion was detected in Cisd2^{-/-} cardiac muscle. Arrow heads indicate mitochondria with partial destruction of outer or inner membranes. (10C) This representative TEM micrograph of a Cisd2^{-/-} sciatic nerve shows an AVd present in the axonal component of a myelinated axon; in addition, many AVds were observed in the cytoplasm of Schwann cell. (10D) An early or initial autophagic vacuole (AVi) was observed in the cytoplasm of Schwann cell of a myelinated axon of Cisd2^{-/-} sciatic nerve. (10E) A myelinated axon with disintegrating myelin sheath (*) and an AVd present in the cytoplasm of Schwann cell of the Cisd2^{-/-} sciatic nerve. All of the samples were prepared from 2-week old Cisd2^{-/-} mice.

FIG. 11 shows autophagy appearing to be induced by damaged mitochondria in muscles and nerves of the Cisd2^{-/-} mice. (11A and 11B) Representative TEM micrographs for skeletal and cardiac muscles, respectively, prepared from 12-week (wk) old Cisd2^{-/-} mice. A cluster of autophagic vacuoles and degenerating mitochondria is present between myofibrils (arrows). The yellow arrow head indicates a mitochondrion undergoing destruction of outer membrane. (11C and 11D) Late or degradative autophagic vacuoles (AVd) and an early or initial autophagic vacuole (AVi, arrow head) enclosing a mitochondrion (Mt) were observed in a specimen prepared from brain (cortex) tissue of a 12-week old Cisd2^{-/-} mouse. (11E and 11F) Early or initial autophagic vacuoles (AVi) were detected in the 3-week old Cisd2^{-/-} optic nerve (arrow head). (11G and 11H) Autophagic vacuoles of AVd (arrows) and AVi (arrow heads) were more frequently detected in the axonal component of myelinated axons of the optic nerve in 24-week old Cisd2^{-/-} mice.

FIG. 12 shows percentage of myelinated axons presented in the sciatic nerves showing disintegration of their myelin sheaths and autophagic vacuoles, including AVi and AVd, in their axonal component. There were 3 mice for each group (12A and 12B). (12C) Western blotting to detect the presence of the proteins LC3-I and LC3-II. (12D) Ratios of the LC3-II to LC3-I. There were three mice for each group. *p<0.05; **p<0.005.

FIG. 13 shows food consumption, water drinking, urine and stool generation were measured daily for the Cisd2^{-/-} and wild-type mice from 6- to 8-week old (13A) or from 12- to 14-week old (13B).

FIG. 14 shows Cisd2 which is primarily localized in the outer mitochondrial membrane and Cisd2 deficiency leads to mitochondrial dysfunction. (14A) EGFP-tagged Cisd2 protein is directed to the mitochondria by an N-terminal signal sequence. The EGFP-Cisd2 proteins were expressed in NIH3T3 cells. EGFP-tagged full-length Cisd2 protein was colocalized with MitoTracker Red, whereas deletion of the N-terminal 58 amino acids completely abolished mitochondria localization. When the N-terminal 58 of 36 amino acid sequence was fused to EGFP, this construct was able to redirect EGFP from a nuclear and cytoplasmic localization to the mitochondria. (14B) Subcellular localization of the Cisd2 and Cisd1 proteins analyzed by Western blotting using protein extracts of the mitochondrial (Mito) and cytosolic (Cyto) fractions prepared from skeletal muscles of 12-week old mice. Polyclonal antibody (Ab) against Cisd2 protein (15 kDa) was generated; this antibody cross-reacts with Cisd1 protein (12 kDa). Antibodies against mitochondrial proteins Cisd1 and Hsp60 were used as controls. (14C) Ten micrograms of each submitochondrial fraction prepared from the livers of 4-week old mice were analyzed by Western blot using antibodies against Cisd2 and known mitochondrial marker proteins. Outer membrane (OM) marker: VDAC-1, voltage-dependent anion channel-1; inner membrane (IM) marker: ATPSB, complex V beta subunit; matrix marker: PDH, pyruvate dehydrogenase. MP, microplast (inner membrane and matrix); IMS, intermembrane space. (14D) Impaired mitochondrial respiration in the skeletal muscle of 4-week old Cisd2^{-/-} mice. Representative oxygraphs of the mitochondria after adding first glutamate-malate and then ADP into the closed chamber of the oxygen meter. (14E) Respiratory activity was expressed as oxygen consumption rate (nmol O2/min/mg mitochondria) in the resting state, for glutamate-malate supported respiration and for ADP activated respiration. A significant decrease in oxygen consumption was detected in the Cisd2^{-/-} mitochondrial samples (n=4) compared with wild-type samples (n=3). (14F) Respiratory control ratio (O2 consumption rate after ADP addition/O2 consumption rate after glutamate-malate addition) was significantly lower in the Cisd2^{-/-} mitochondria. (14G) Comparison of electron transport activities of the respiratory enzyme complexes of mitochondria prepared from the skeletal muscles of 4-week old Cisd2^{-/-} (n=4) and wild-type mice (n=4). NCCR activity: measurement of NADH cytochrome c reductase activity, which represents complex I-III; SCCR activity: measurement of succinate cytochrome c reductase activity, which represents complex II-III; CCO activity: Cytochrome c oxidase activity, which represents complex IV. *p<0.05; **p<0.005.

FIG. 15 shows Subcellular localization of Cisd2 protein in skeletal muscle: A small portion of the Cisd2 protein was co-localized with the endoplasmic reticulum (ER)/sarcoplasmic reticulum (SR) markers in the microsomal fractions of skeletal muscle. (15A) Western blot analysis of homogenate (H), pellet (P; nuclei and mitochondria) and post-mitochondrial supernatant (PMS; SR and other cytosolic components) of skeletal muscle using antibodies against Cisd2 and mitochondrial (Mito) markers VDAC-1 and Hsp60. (15B) Quantification of the Cisd2 protein levels in the P and PMS fractions detected by Western blot. (15C) A microsomal preparation of skeletal muscle was fractionated from PMS on a sucrose density gradient and the microsomal fractions (F1-F5) were examined together with the cytosolic supernatant (S) by immunoblot using antibodies against Cisd2 and ER markers Calnexin and Grp78.

FIG. 16 shows Optic nerve degeneration and impaired glucose tolerance in Cisd2^{-/-} mice. (16A) A representative TEM micrograph showing a late or degradative autophagic vacuole (AVd) detected in the axonal component of a myelinated axon of the optic nerve in 24-week old Cisd2^{-/-} mice. The white arrow indicates a disintegrating myelinated axon. (16B) Percentage of myelinated axons of the optic nerves containing autophagic vacuoles, including AVi and AVd, in the axonal component. There were 3 mice for each group; wk, week. (16C and 16D) Blood glucose levels and plasma insulin levels, respectively, before (0 min) and after the glucose load at the indicated time points. Oral glucose (1.5 g/kg body weight) tolerance tests were performed on 12-week old Cisd2^{-/-} and wild-type mice, all of which had a C57BL/6 genetic background. Blood samples were collected to determine the mice's blood glucose levels and plasma insulin levels. (16E) Insulin (0.75 unit/kg body weight) tolerance tests were performed on 12-week old Cisd2^{-/-} and wild-type mice. There were three mice in each group and three independent measurements were carried out on each mouse. *p<0.05; **p<0.005. (16F) IHC staining of insulin in the beta-cells of pancreatic islets using tissue sections prepared from 12-week old Cisd2^{-/-} and wild-type mice.

FIG. 17 shows the study of resveratrol (RES) in the animal model. (17A) Oral administration of resveratrol (30 mg/kg/day) to Cisd2 knockout mice from 4- to 12-week old. (17B) To analyze the body weight of the Cisd2 knockout mice after resveratrol treatment and compare to 4-week old wild-type mice. Values represent mean±s.d from at least three male samples. Asterisks indicate statistically significant differences compare with untreated control. (*, P<0.05)

FIG. 18 shows resveratrol treatment which has partial rescue on muscle and neuron degeneration of the Cisd2 knockout mice. (18A-F) H&E staining of transverse sections of skeletal muscle dissected from the 12-week old, male Cisd2 knockout mice with (18A) untreated control, (18B) resveratrol or (18C) H₂O treatment, and (18D-F) relative wild-type control. Black arrowheads indicate degenerated transverse fibers. Blue arrows indicate angular fibres which are the evidences of muscle atrophy caused by neuron degeneration. (18G) The standard quantification score of muscle atrophy. (18H) The quantification of degenerative muscle fiberin under resveratrol treated or not. About 1000 muscle fibre in random fields of H&E staining slides were examined for each mouse. Values represent mean±s.d from at least three male samples. Asterisks indicate statistically significant differences compare with untreated control. (*, P<0.05; **, P<0.01) Ultrastructure of (18I-K) skeletal and (18L-N) cardiac muscle dissected from Cisd2 knockout mice with resveratrol treatment, Cisd2 knockout mice without resveratrol treatment, and 12-week old wild-type, respectively. White arrows indicate degenerated mitochondria. (18O-T) A myelinated nerve fiber from the sciatic nerve of Cisd2 knockout mice with resveratrol treatment, Cisd2 knockout mice without resveratrol treatment, and 12-week old wild-type, respectively. The axon is enveloped with myelin sheath (MS) formed by fusion of many layers of Schwann cell plasma membrane. Myelin sheath degeneration (asterisks) was detected only in the Cisd2 knockout mice. N, nucleus of Schwann cell. Yellow arrowhead indicates debris of a breakdown myelinated fiber. (18U) The standard quantification score of axon and myelin sheath degeneration. (18V) The quantification of degenerative sciatic nerve in wild-type, Cisd2 knockout and Cisd2 knockout mice with resveratrol treatment, respectively. About 500 axons in random fields of TEM's grids were examined for each mouse.

### DETAILED DESCRIPTION OF THE INVENTION

### Definition

The Cisd2-knockout mouse used in the present invention is equal to the Cisd2-knockout mouse in U.S. Application No. 60/849,089.

The term “Cisd2” as used herein means Mus musculus CDGSH iron sulfur domain 2, and the orthologous genes including Gret, ZCD2, Miner1, Noxp70, AI848398, 1500009M05Rik, 1500026J14Rik, 1500031D15Rik, and B630006A20Rik.

The present invention applies a mouse genetics approach and demonstrated that Cisd2 is involved in mammalian lifespan control and plays an essential role in mitochondrial integrity. Cisd2 deficiency causes mitochondria-mediated phenotypic defects in mice. Furthermore, cell culture and biochemical investigations revealed that Cisd2 is a mitochondrial protein. Additionally, Cisd2 knockout mice exhibit many clinical manifestations of WFS patients including early-onset degeneration of central (e.g. optic) and peripheral (e.g. sciatic) nerves and premature death, as well as impaired glucose tolerance. This study therefore provides an animal model for mechanistic understanding of WFS, specifically WFS2, pathogenesis.

The present invention recapitulates a more extensive set of early senescence associated features of human premature aging than those previously described. As such, the present invention provides an extremely useful model to elucidate premature aging or WFS2 disease in human.

Furthermore, the present invention offers an in vivo system to screen for agents in ameliorating the patho-physiological effects of premature aging or WFS2 disease.

A mutant animal of the present invention can be any non-human mammal, preferably a mouse. A mutant animal can also be, for example, any other non-human mammals, such as rat, rabbit, goat, pig, dog, cow, or a non-human primate. It is understood that mutant animals having a disrupted Cisd2 gene, as disclosed herein, or other mutant forms that eliminate the expression of Cisd2, can be used in methods of the invention. Thus, the mutant animal loss of all or a part of the Cisd2 gene function is due to a disruption of the Cisd2 gene

The present invention provides a line of genetically engineered mice either heterozygous (referred to as Cisd2^{+/-}) or homozygous (referred to as Cisd2⁴⁻) for the disrupted endogenous Cisd2 gene. This gene may be mutated by disrupting one or more of its exons by heterologous DNA sequences such as an HPRT cassette using standard molecular biological techniques. In addition, any mutant forms that eliminate the expression of Cisd2 can be used. The resulting Cisd2^{-/-} mice exhibit a range of phenotypes similar to those of human aging including many physical or biochemical manifestations as detailed below. As such, these mice, Cisd2^{+/-} and Cisd2^{-/-} included, can be used as a model system to help delineate the molecular mechanisms underlying human premature aging or WFS2 disease.

The present invention also provides a cell or cell line from the Cisd2 knockout mouse, wherein the cell or cell line contains a targeted disruption in Cisd2 gene in which Cisd2 exon 3 has been disrupted. The cell or cell line is an undifferentiated cell which is selected from the group consisting of a stem cell, embryonic stem cell oocyte and embryonic cell.

The present invention further demonstrates a method of screening for agents useful in treating or preventing premature aging or WFS2 disease associated phenotypes or delaying the onset of premature aging consisting of administering candidate compounds to the Cisd2^{-/-} mice or the cell or cell line derived from the Cisd2^{-/-} and screening for the desired therapeutic effects.

The method for identifying a target gene having altered expression in a mutant Cisd2 mouse involves comparing the expression of one or more genes in a mutant mouse having a disrupted Cisd2 gene with the expression of said one or more genes in a wild type animal to identify a gene having altered expression in said mutant mouse, thereby identifying a target gene having altered expression in a mutant Cisd2 mouse.

As described in Example 7, Cisd2 mutant mice exhibited altered expression of genes in comparison to wild type mice in addition to other phenotypes described. For instance, Cisd2 knockout mice are characterized by decreased expression of BDNF (brain-derived neurotrophin factor). The altered expression of BDNF gene, as well as other genes having altered expression in a mutant Cisd2 mouse, indicates that Cisd2 normally regulates the expression of these genes in wild-type mice. Thus, these represent genes that can be modulated to reverse, or at least partially reverse, the physiological and biochemical characteristics of a Cisd2^{-/-} phenotype. For example, restoring the expression of one of these Cisd2 regulated genes having altered expression in a mutant Cisd2 mouse to a level that can result in reversed phenotypes can be contemplated. Therefore, a compound that exhibits the said effect is a potentially useful therapeutic compound for treatment of premature aging associated phenotypes or possibly delaying the onset of premature aging.

As such, the present invention provides methods for identifying target genes having altered expression in a mutant Cisd2 mouse, as well as methods for identifying a compound that restores a target gene having altered expression in a mutant Cisd2 mouse to a level of expression achieving the desired therapeutic effect.

The methods of the invention for identifying a target gene having altered expression in a mutant Cisd2 mouse can involve comparing the expression of one or more genes contained within one or more organs of the mutant Cisd2 mice.

The method for identifying a compound that restores a target gene having altered expression in a mutant Cisd2 mouse to a therapeutic level of expression involves (a) contacting a target gene having altered expression in a mutant Cisd2 mouse with a test compound; (b) determining expression of said target gene, and (c) identifying a compound that modulates expression of said target gene to a level of expression consistent with a wild type level of expression.

The methods of the invention for screening for a compound that restores a target gene having altered expression in a mutant Cisd2 mouse to a more normal level of expression-involve contacting a sample exhibiting altered expression of a target gene characteristic of a mutant Cisd2 mouse with a test compound. A test compound can be any substance, molecule, compound, mixture of molecules or compounds, or any other composition which is suspected of being capable of restoring an expression level of a target gene to a more normal level.

Additionally, a test compound can be pre-selected based on a variety of criteria. For example, suitable test compounds having known modulating activity on a pathway suspected to be involved in a mutant Cisd2 phenotype can be selected for testing in the screening methods. Alternatively, the test compounds can be selected randomly and tested by the screening methods of the present invention.

A level of protein expression corresponding to a gene expression level also can be determined, if desired. A variety of methods well known in the art can be used to determine protein levels either directly or indirectly.

The methods of the invention for identifying a compound that restores a target gene having altered expression in a mutant Cisd2 mouse to a more normal level of expression can involve determining an activity of a target gene. The activity of a molecule can be determined using a variety of assays appropriate for the particular target. A detectable function of a target gene can be determined based on known or inferred characteristics of the target gene.

For use as a therapeutic agent, the compound can be formulated with a pharmaceutically acceptable carrier to produce a pharmaceutical composition, which can be administered to the individual, which can be a human or other mammal.

The methods of the invention can advantageously use cells isolated from a homozygous or heterozygous Cisd2 mutant mouse for a desired purpose. For example, these cells can be used as an in vitro method to screen agents for treating or preventing premature aging or WFS2 disease associated phenotypes or the onset of premature aging or the disease. In such a method, a compound is contacted with a cell having disrupted Cisd2 expression, and screen for modulation of the target gene as described above.

Thus, the invention provides methods of screening a large number of compounds using a cell-based assay, for example, using high throughput screening, as well as methods of further testing compounds as therapeutic agents in an animal model using the Cisd2 mutant mice.

The present invention is further directed to cell lines derived from the Cisd2^{+/-}, or Cisd2^{-/-} mice. These cell lines are useful in studying senescence at the cellular level and in drug screening assays. Cell lines derived from the brain, kidney, lung, stomach, intestine, spleen, heart, adipose, heart and liver tissues are especially useful in these applications.

In a preferred embodiment, the present invention is related to a Cisd2 knockout mouse with phenotype comprising mitochondrial breakdown and dysfunction, wherein Cisd2 is defined as SEQ ID NO. 1. In a more preferred embodiment, the mouse of the present invention has the phenotype comprises nerve demylination and neuron degeneration, cardiac and skeletal muscle degeneration, reduced body weight, prominent eyes and protruding ears, osteopenia, lordokyphosis, abnormal pulmonary function, opacity of the cornea, or skin atrophy and graying.

In a preferred embodiment, the Cisd2 gene is knockout by recombination with homologous nucleotide sequence. In a more preferred embodiment, knockout occurs in Cisd2 exon 3.

In a preferred embodiment, the mouse of the present invention has Cisd2 knockout steps comprising:

(a) an additional copy of a Cisd2 gene fragment consisting of a portion of intron 1, the entire exon 2, and a portion of exon 3 of the Cisd2 gene;
(b) a positive puromycin selection marker;
(c) a non-functional 3′-HPRT cassette; and
(d) a loxP site.

In a preferred embodiment, the present invention is related to a mouse model of Wolfram Syndrome 2 (WFS2) disease consisting of a Cisd2 knockout mouse aforementioned. In a more preferred embodiment, the present invention is related to a mouse model which is applied to screen a candidate agent for preventing or treating WFS2 disease.

The present invention also relates to a transgenic knock-out mouse whose genome comprises a homozygous disruption in its endogenous CDGSH iron sulfur domain 2 (CISD2) gene, wherein said CISD2 is defined as SEQ ID NO. 1, and wherein said homozygous disruption results in said transgenic knockout mouse exhibiting decreased levels of CISD2 as compared to a wild-type mouse, said mouse showing symptoms similar to the features of premature aging, said features consisting of optic atrophy, neurological features, ataxia, cardiac and skeletal muscle degeneration and nerve demyelination and neuron degeneration.

In a preferred embodiment, the optic atrophy of the transgenic knock-out mouse is a phenotype of progressive degeneration of optic nerve starts from age 3-week of the transgenic knock-out mouse. In a preferred embodiment, the ataxia of the transgenic knock-out mouse is a phenotype of progressive degeneration of sciatic nerve and unsteady gait starts from age 2-week of the mouse. In a preferred embodiment, the feature of cardiac and skeletal muscle degeneration in the transgenic knock-out mouse starts from age 3-week of the mouse. In another preferred embodiment, the feature of nerve demyelination and neuron degeneration in the transgenic knock-out mouse starts from age 3-week of the mouse.

The following examples are given for the purpose of illustrating various embodiments of the invention and are not meant to limit the present invention in any fashion.

### EXAMPLE

### Example 1

### Expression Analysis of the Mouse Cisd2 Gene

The mouse Cisd2 (SEQ ID NO. 1) was identified as the putative ortholog based on the remarkable protein sequence similarity (96% identity) to the human gene, Cisd2, located in the region where the longevity locus was previously mapped. It was then engineered and disrupted to understand its role in longevity in the present invention.

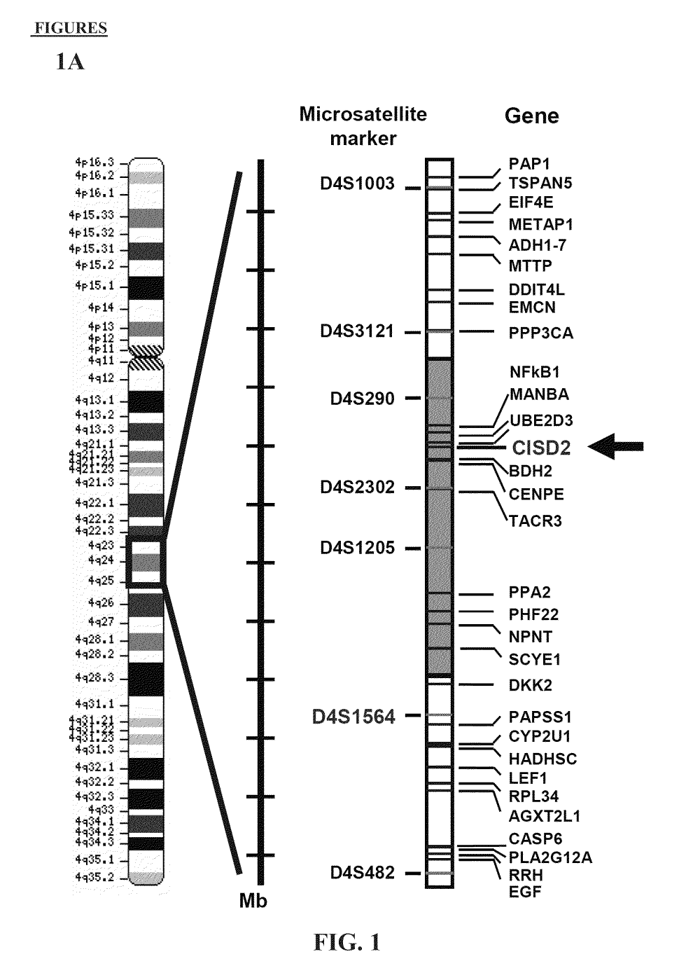

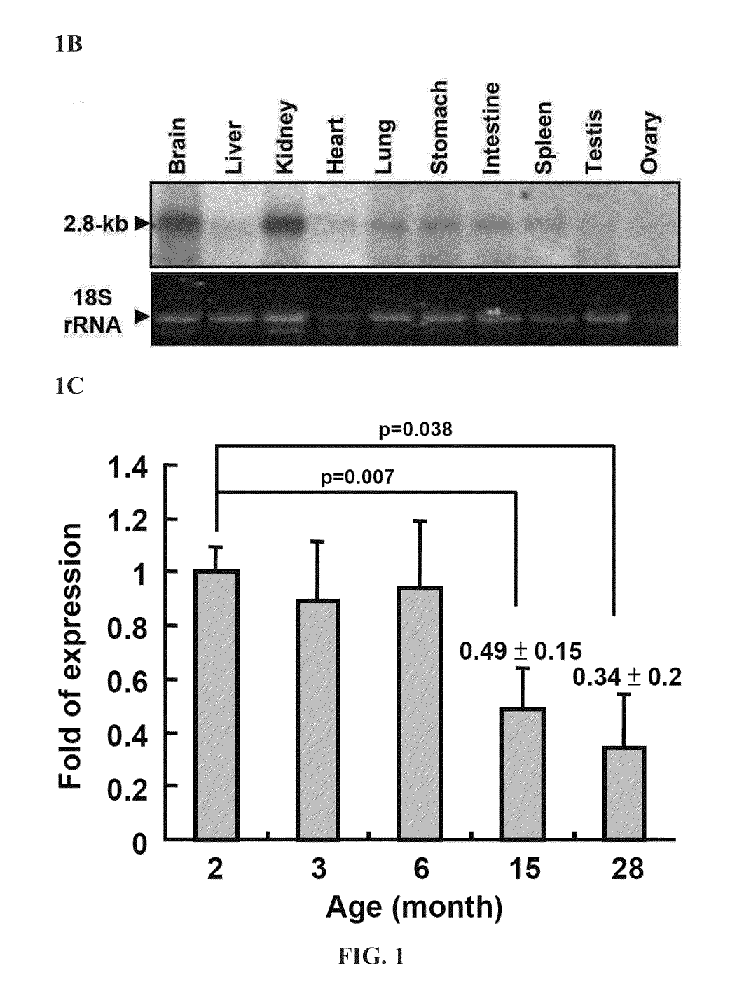

The expression pattern of the Cisd2 gene was characterized by examining the relative levels of mRNA present in adult mouse tissues (FIG. 1B). A band of 2.8-kb was detected at higher levels in brain and kidney. A similarly sized band but at lower levels was detected in lung, stomach, intestine, and spleen. There were much lower levels in liver, heart, testis and ovary. Northern blot analysis showed that Cisd2 is a widely expressed gene in mice. Quantitative real-time RT-PCR revealed that expression levels of Cisd2 decrease in an age-dependent manner in naturally aged mice (FIG. 1C).

### Example 2

### Generation of Cisd2^{+/-} and Cisd2^{-/-} Mice

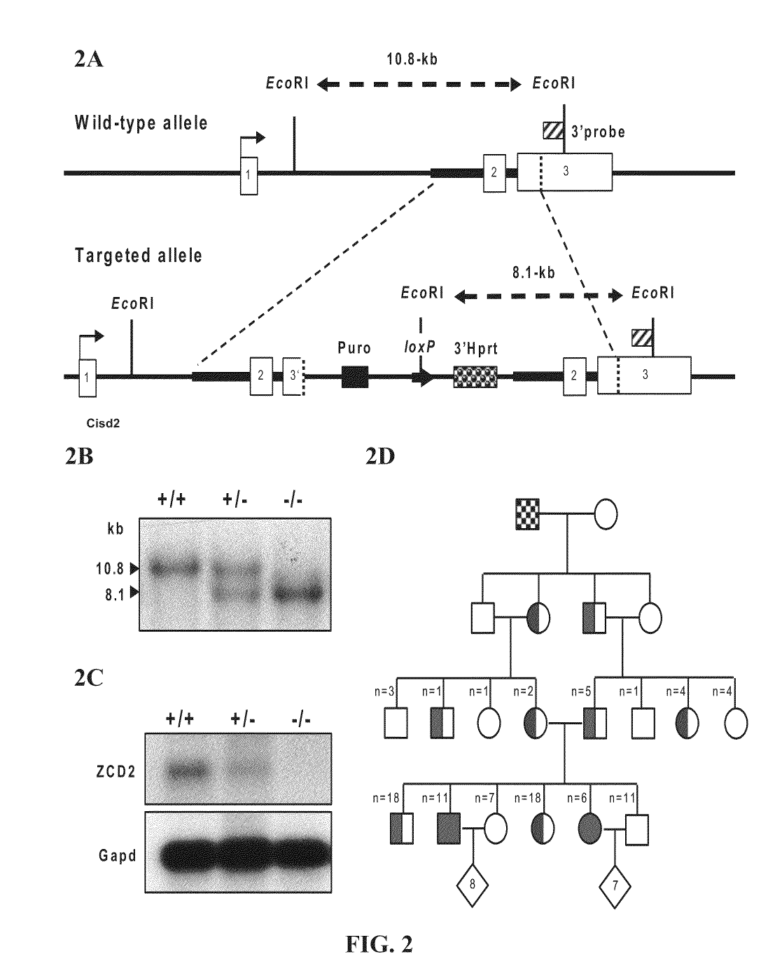

FIG. 2A showed the strategy used to create the targeted mutation. A BAC library (Research Genetics Inc.) derived from mouse strain C57BL/6 genomic DNA was screened with two pair of primers (SEQ ID NO. 2-5) designed from conserved sequence of Cisd2 gene between human (Hs.29835) and mouse (Mm.41365). The mouse of BAC clones, YM-BAC-210J1 and YM-BAC-412J13, were identified and purified. Several genomic DNA fragments covering the mouse Cisd2 gene were subcloned from YM-BAC-210J1. A SpeI-BamHI 6.4 kb DNA fragment, which contains part of intron 1, exon 2 and part of exon 3, was further subcloned and used as homologous recombination arm for construction of an “insertion-type” targeting vector for the mouse Cisd2 gene. The SpeI-BamHI 6.4 kb fragment was inserted into the EcoRV site of the pG12 vector, which contained the puromycin selection cassette, a loxP site and 5′ truncated Hprt gene. The Cisd2 targeting vector, pG12/ApaI(−)-SpB6.4, was amplified and linearized within the homologous recombination arm using ApaI.

The linearized targeting vector was electroporated into 129/SvEv embryonic stem (ES) cells. Selection medium containing puromycin and gancycloviour was applied 24 h after electroporation and maintained for 7 days. Resistant colonies were selected and re-seeded onto the feeder layer in a 96-well plate. DNA extracted from individual ES clone was isolated and detected by Southern blot analysis. The 3′ flanking probe used was a 1.7 kb BamHI-EcoRI fragment from exon3 (FIG. 2A).

The targeted ES cells were injected into C57BL/6 blastcysts and reimplanted into pseudopregnant female mice. Chimeric male mice were bred with C57BL/6 female. Genomic DNA was isolated from tail samples of the appropriate agouti progeny using proteinase K/SDS digestion and phenol/chloroform extraction method. Isolated DNA samples were further analyzed by Southern blot for germline transmission. The analysis confirmed the presence of both the endogenous and the disrupted alleles in the F1 heterozygotes. The heterozygous mice were intercrossed, and their offspring were genotyped.

Genotypes of offspring from heterozygous breeding demonstrated normal Mendelian ratios of homozygous (−/−), heterozygous (+/−) and wild-type (+/+). Fertility test of the Cisd2^{-/-} males and females exhibited normal reproductive capability.

Southern blot analysis showed that the genomic DNA digested with EcoRI and hybridized with a probe shown in FIG. 2A gave the signals expected from the wild-type (+/+), heterozygous (+/−), and homozygous-null (−/−) animals (FIG. 2B).

Northern blot of total RNA prepared from the brain tissue of wild-type (+/+), heterozygous (+/−), and Cisd2-null (−/−) mice was probed with the ³²P-labeled fragment identical to that used in Southern blot analysis. The probe detected a 2.8-kbp RNA band in samples from the wild-type and heterozygous but not from the homozygous animals. Hybridization of the same filter, after stripping of the Cisd2 probe, with a mouse glyceraldehyde-3-phosphate dehydrogenase (Gapd) probe confirmed that equal amounts of RNA were loaded on the gel.

### Example 3

### Early Senescence Including Reduced Life Span and Growth Retardation in Cisd2^{-/-} Mice

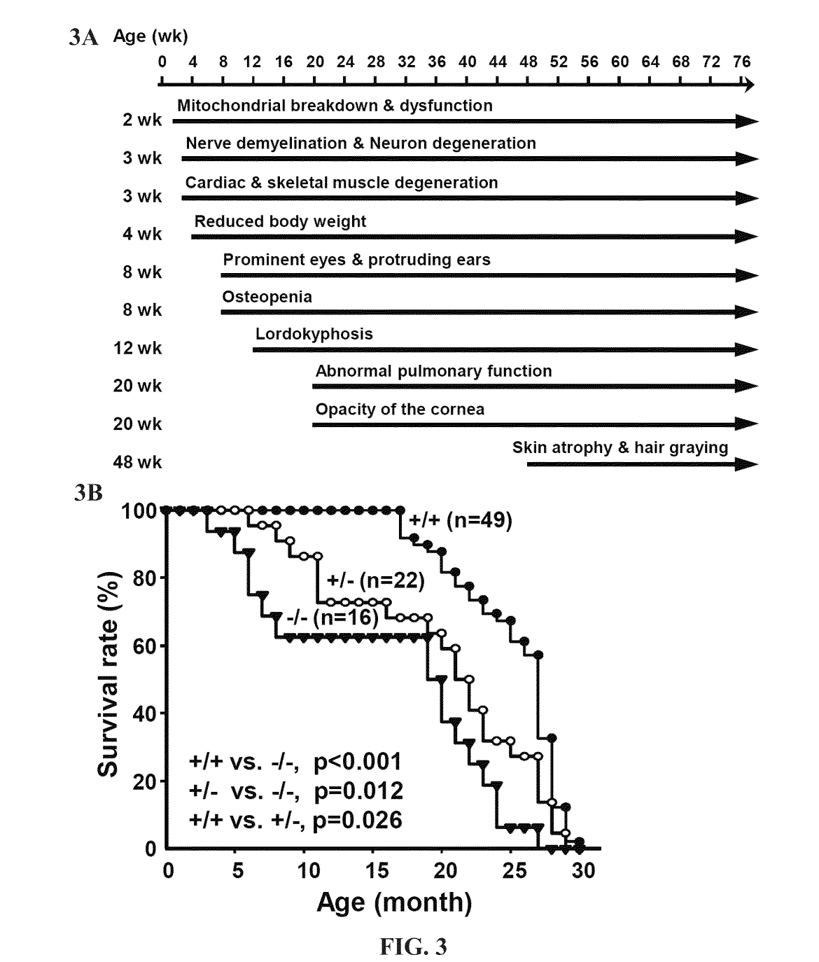

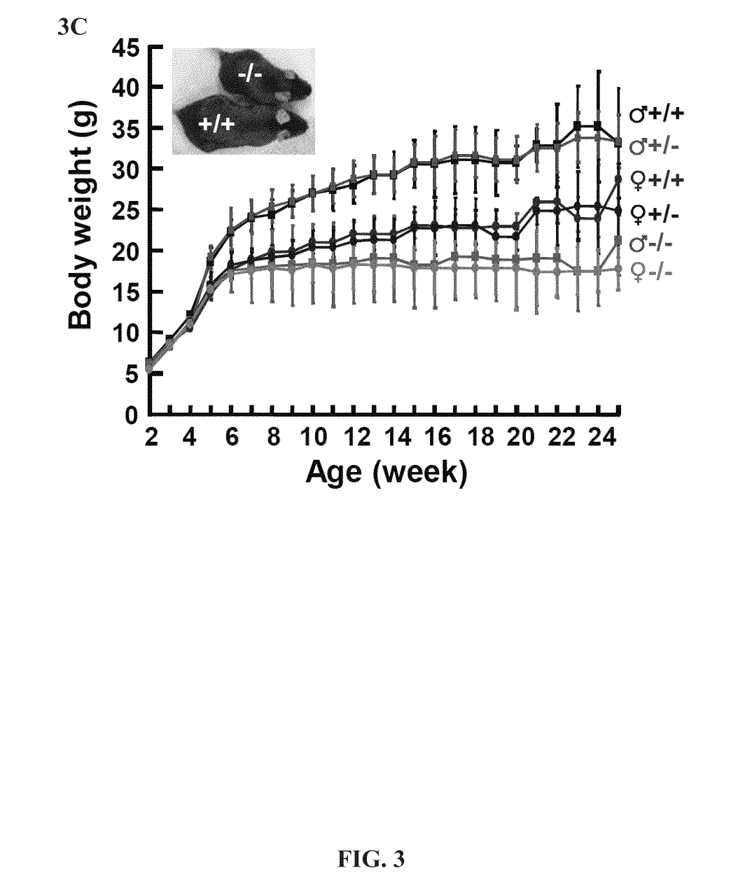

Up to 3 weeks of age, Cisd2^{-/-} mice appeared morphologically identical to their Cisd2^{+/+} littermates. However, starting around week 3, all of the Cisd2^{-/-} mice started to display a wide range of senescence associated phenotypes shown in FIG. 3A with the time of onset indicated for each phenotype. Early senescence was accompanied by a shortened lifespan when survival of the various genotypes was examined and there appeared to be signs of haplo-insufficiency for Cisd2 in view of the slightly lower survival rate for the heterozygous (Cisd2^{+/-}) mice (FIG. 3B). Furthermore, growth retardation and a smaller somatotype were clearly evident; it appeared that there was almost no growth after 5-week old in the Cisd2^{-/-} mice (FIG. 3C).

### Example 4

### Eye and Cutaneous Phenotypes in Cisd2^{-/-} Mice

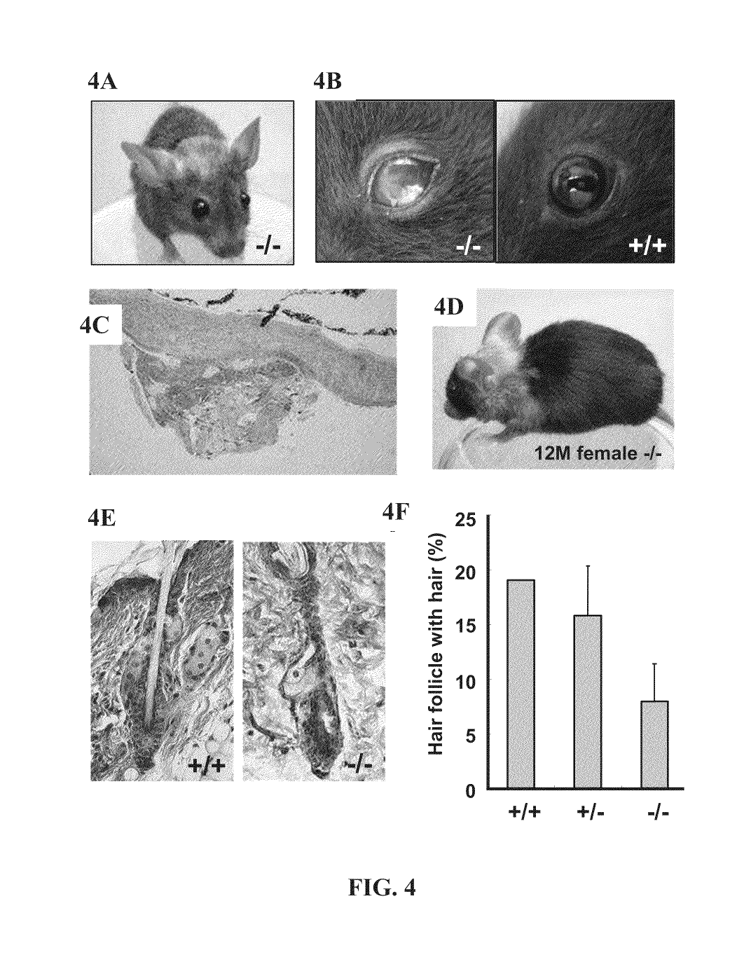

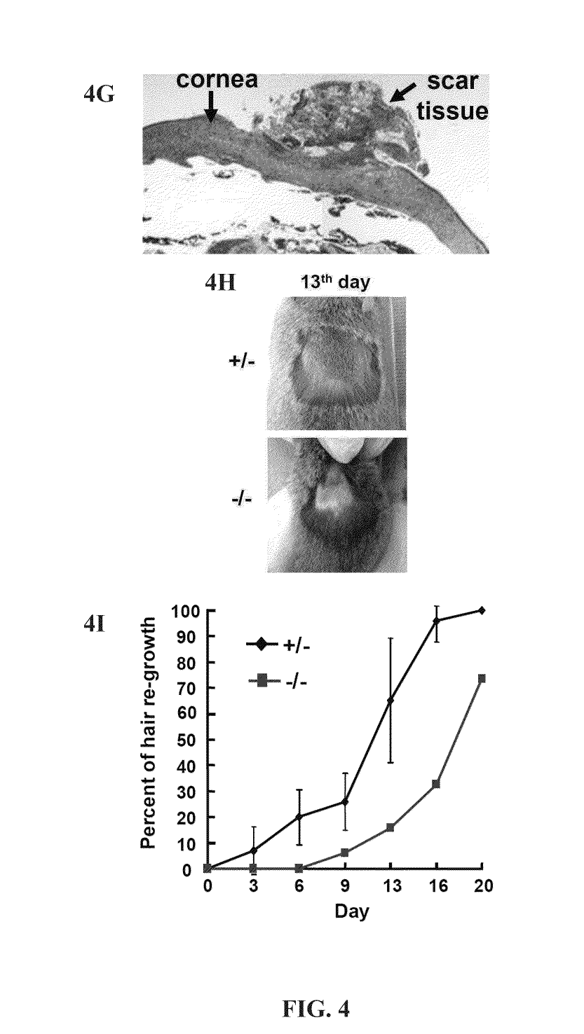

Starting at 8-week old, the Cisd2^{-/-} mice began to acquire a set of aged appearance remarkably similar to those displayed by patients with Hutchinson-Guilford progeria syndrome. These included prominent eyes and protruding ears (FIGS. 4A, E, and F). Ocular abnormalities were observed as the Cisd2^{-/-} mice developed opaque eyes and blindness, which was accompanied by cornea damage at 20-week old (FIG. 4B). There was also early depigmentation in the fur (FIG. 4D) of 48-week-old Cisd2^{-/-} mice where no depigmentation was observed in the aged matched Cisd2^{+/+} littermates. Ocular abnormalities were also observed where the 20-week-old Cisd2^{-/-} mice had opaque eyes similar to symptoms of cataracts and became blind with accompanying cornea atrophy (FIG. 4B). The opacity of the cornea was investigated and histological analysis found collagen deposition that appeared to correlate with the observed ocular phenotype. Histopathological examination revealed that the opacity of the cornea was due to debris deposition in the scar tissue outside the cornea (FIG. 4G). A decrease in the hair re-growth rate was also observed in the Cisd2^{-/-} mice (FIGS. 4H and 4I).

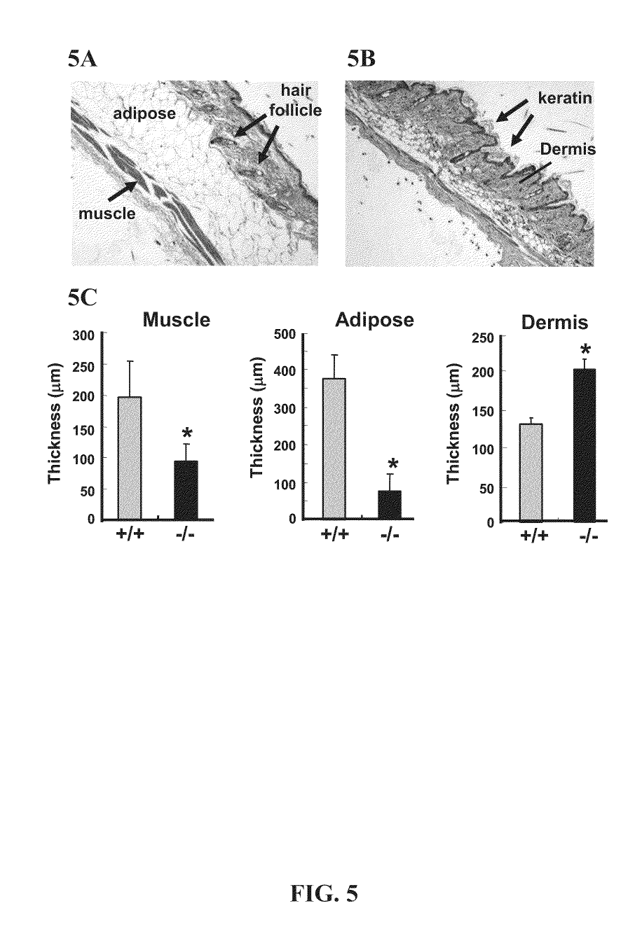

Two anatomical characteristics commonly seen in aged human skins were reduced dermal thickness and subcutaneous adipose. Consistent with those features in human, the skin of 48-week-old Cisd2^{-/-} mice exhibited phenotypes of massive hyperkeratosis, significant decrease of subcutaneous fat and muscle, and noticeably thickened dermis with expanded surface (FIG. 5B) compared with those of age-matched wild-type mice (FIG. 5A). Though abundantly presented in skin of 48-week-old wild-type mice, subcutaneous adipose cells were nearly absent in that of age-matched Cisd2^{-/-} mice. Quantitative analysis confirmed mean thicknesses of muscle and adipose layer for skin of 48-week-old Cisd2^{-/-} mice was considerably reduced compared with those for skin of age-matched wild-type mice while there was a concomitant increase in the mean thickness of the dermis layer.

Tissue sections of the dorsal skin were stained with H&E and Masson's trichome staining. The thicknesses of the dermal, adipose and muscle layers were quantified by random measurements of the length of individual skin samples using SPOT Imaging Software Advance (DIAGNOSTIC Instruments Inc.).

### Example 5

### Abnormal Skeleton and Pulmonary Functions in Cisd2^{-/-} Mice

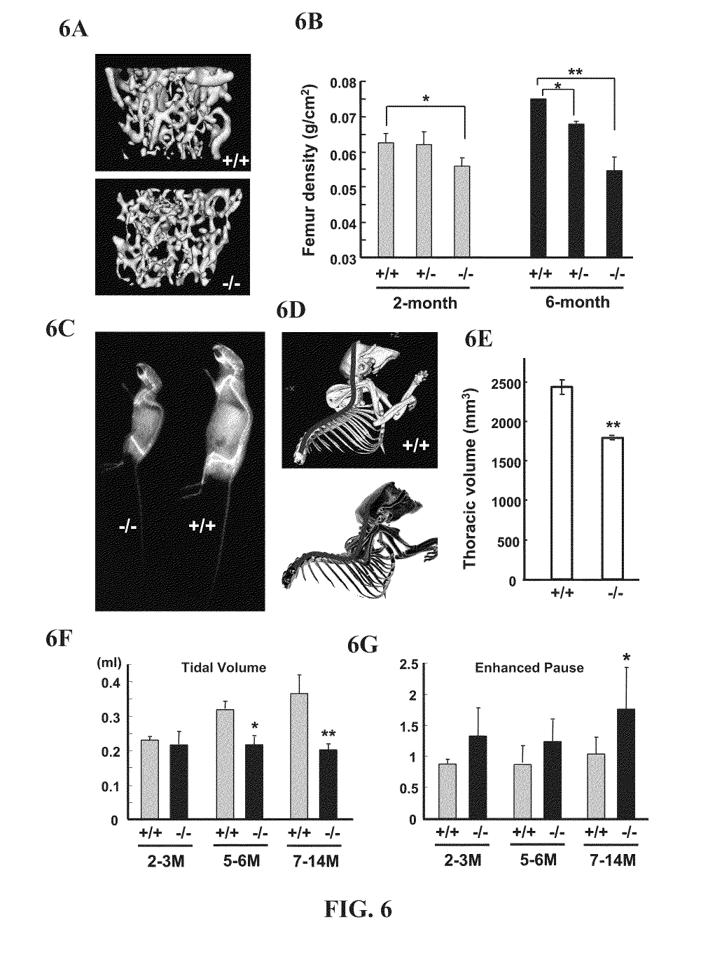

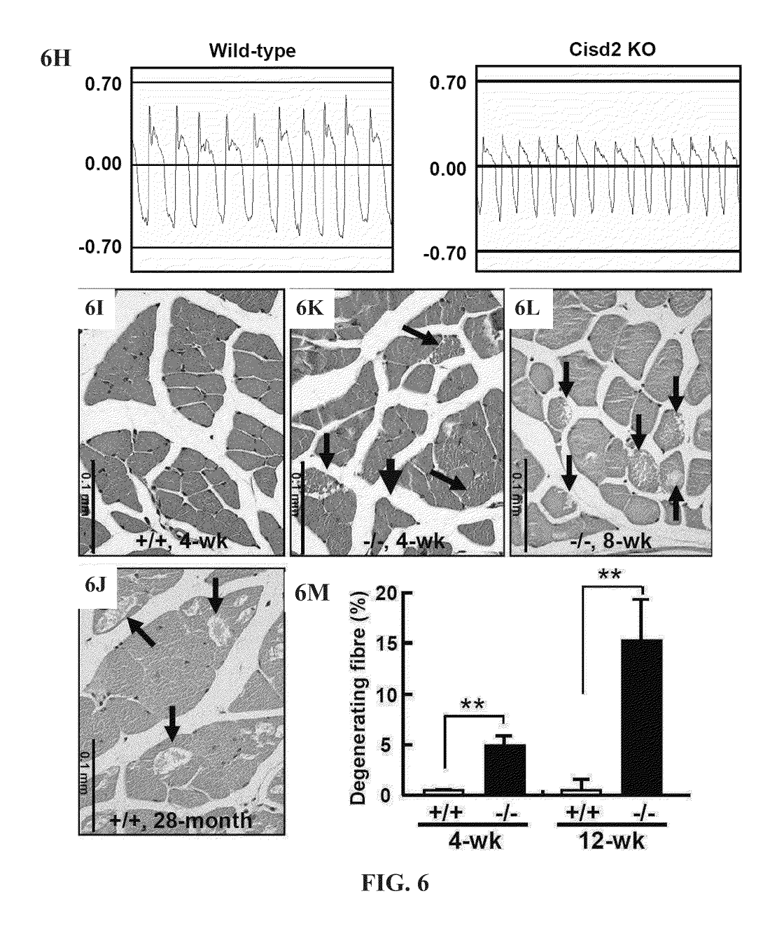

Micro-computer tomography analysis detected a decrease of femur density in the 8-week-old Cisd2^{-/-} mice compared with that of the age-match wild-type mice while the trabeculae of the femur in Cisd2^{-/-} mice were noticeably thinner (FIG. 6A). Interestingly, a decrease of femur density started to emerge in 24-week-old Cisd2^{+/-} mice while a progressively more severe phenotype was observed in the age-matched Cisd2^{-/-} mice (FIG. 6B). This showed, in addition to what was observed in life span evaluation, an apparent Cisd2 with respect to femur density but was only obvious after 24 weeks of age.

The bone samples of wild-type and Cisd2^{-/-} mice were fixed in 10% buffered formalin phosphate, stored in 70% ethanol and examined by eXplore Locus SP Pre-Clinical Specimen MicroCT (GE Healthcare). Whole-body and femur scans were performed in the axial plane mounted in a cylindrical sample holder. The three-dimensional images of bones reconstructed from MicroCT scanning slices used to qualitatively evaluate bone structure and morphology. The quantitative data of bone tissue were separated from those for marrow and soft tissue and were analyzed by eXplore MicroView v. 2.0 Software Guide (GE Healthcare).

While showing no detectable skeletal abnormalities up to 8 weeks of age, radiographs of 12-week-old Cisd2^{-/-} mice already displayed significant lordokyphosis (curvature of the spinal column) (FIG. 6C), which resulted in a decrease in mean thoracic volume for them compared with that for the age-matched wild-type mice (FIGS. 6D and 6E). Consequently, the skeletal abnormality affected various respiratory parameters as measured by plethysmography (FIGS. 6F and 6G) and led to abnormal pulmonary functions. These features, including decrease in femur density and lordokyphosis, were manifested in aged humans.

Indeed, the present invention observed decreases in various respiratory parameters as measured by plethysmography after 20-week old in the Cisd2^{-/-} mice (FIG. 6F-H).

Respiratory parameters were measured in conscious mice with three genotypes by using plethysmography chambers where the mouse body was enclosed in a sealed chamber while the head was free. Thoracic movements were measured by pressure transducers that were linked to a Buxco amplifier system and respiratory parameters, then captured and analyzed by the Notocord HEM data acquisition system. Upon placement of the mice into the plethysmography chambers, tidal volume (TV) was determined 10 min at unrestrained condition. The formula for calculating Penh (Enhanced Pause) was: PEF/PIF×(Te/Rt-1), Where Te=Expiratory time, Rt=Relaxation time, PEF was Peak Expiratory Flow, and PIF was Peak Inspiratory Flow.

### Example 6

### Muscle Atrophy and Loss of Adipose Tissue in Cisd2^{-/-} Mice

Muscle degeneration was detectable at 3-week old in the Cisd2^{-/-} mice. There was a progressive degeneration of muscle fibres and the magnitude of the degeneration exacerbated with age (FIG. 6I-M). In addition, angular fibres, which were an indicator of muscle atrophy caused by neuron degeneration, could be observed in the Cisd2^{-/-} mice (FIG. 6K).

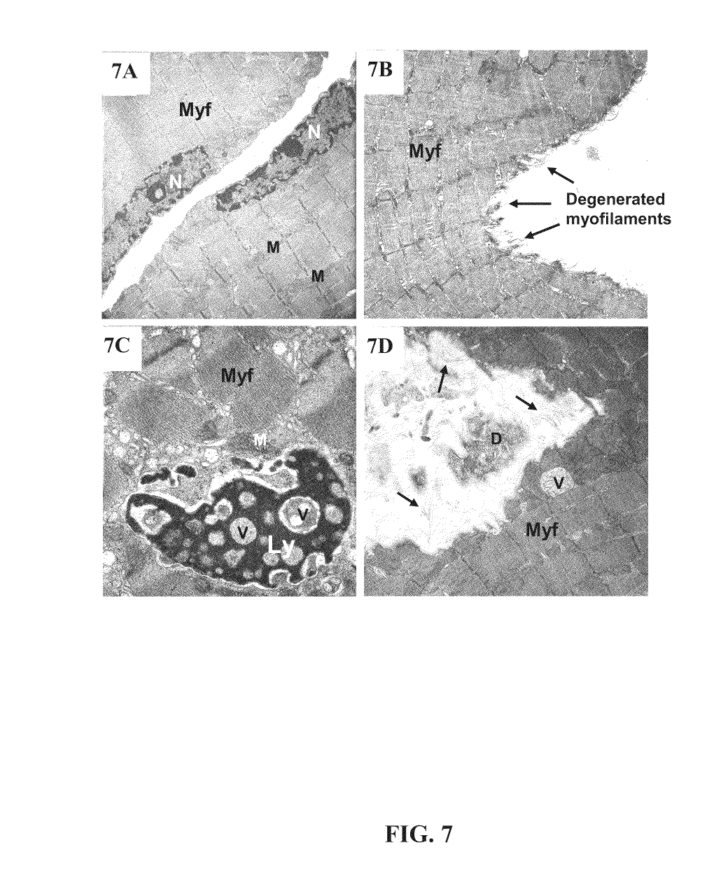

To understand the basis for the morphological abnormality in the longitudinal fibers in Cisd2^{-/-} mice, ultrastructure of muscle cells were examined by electron microscopy. Muscle tissues from wild-type and Cisd2^{-/-} mice were fixed in a mixture of glutaraldehyde (1.5%) and paraformaldehyde (1.5%) in phosphate buffer at pH 7.3. These were postfixed in 1% OsO₄, 1.5% potassium hexanoferrate, rinsed in cacodylate and 0.2 M sodium maleate buffers (pH 6.0), and block-stained with 1% uranyl acetate. Following dehydration, tissues were embedded in Epon and were ready for transmission electron microscopy. Degenerated myofilaments indicated by arrows (FIG. 7B) were separated from a muscle cell in 8-week-old Cisd2^{-/-} mice while myofilaments remain intact in age-matched Cisd2^{+/+} mice (FIG. 7A). Myofibrils of a striated muscle cell were engulfed by lysosomes. There were many digestive vacuoles in lysosome (FIGS. 7C and 7D). Consistent with other phenotypes observed, both muscle atrophy and loss of adipose tissues were hallmarks of human aging.

### Example 7

### Myelin Sheath and Axon Degeneration and Reduction of Brain BDNF in Cisd2^{-/-} Mice

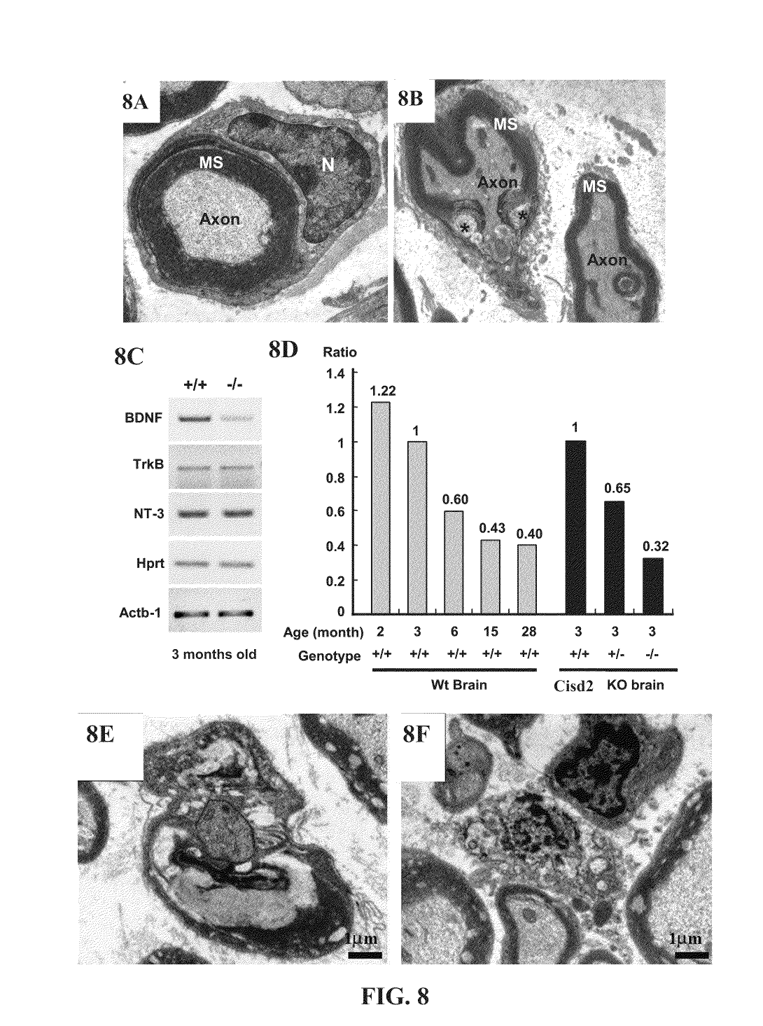

Since myelin sheath degeneration was one of the clinical features in aging, the present invention sought to examine the state of peripheral nerves when Cisd2 gene expression was eliminated. In wild-type mice, the myelinated axons were enveloped with a myelin sheath formed by the fusion of many layers of plasma membrane from Schwann cells (FIG. 8A). However, considerable disintegration of the myelin sheath and degeneration of axon was detected in the Cisd2^{-/-} sciatic nerves (FIGS. 8E and 8F). Remarkably, there appeared to be considerable demyelination occurring in Cisd2^{-/-} nerves with apparent axonal degeneration when ultrastructure of axons and their myelin sheath were examined (FIG. 8B).

To investigate the effect of Cisd2 on transcription of other genes, select number of genes was examined. Reverse transcription was performed with 2 μg of total RNA and primed with random hexamers and Superscript III reverse transcriptase (Invitrogen Life Technologies). Real-time PCR was carried out on Roche LightCycler 480 Real-time PCR instrument, using TaqMan probe searched at Universal ProbeLibrary (Roche applied science) and LightCycler TaqMan Master (Roche applied science). Cycling profiles for real-time PCR were pre-incubated for 10 sec at 95° C., and carried out 55 cycles of 5 sec at 95° C., 20 sec at 60° C., and 2 sec at 72° C. Fluoresce was acquired on each elongation step during amplification and analyzed with the Light Cycler Software 4.05. Significantly, brain-derived neurotrophin factor gene (BDNF) was found to be down-regulated while the levels of other genes such as TrkB, NT-3, HRPT, and Actb-1 remain unchanged. This correlates with the observation that expression levels of BDNF decreased with age as demonstrated in the 15-month and 28-month-old wild-type mice compared with that in the younger mice (FIG. 8D). Dose-dependent decrease of BDNF mRNA was detected in the heterozygous and homozygous Cisd2 knockout mice. Notably, the expression level of BDNF in the brain of 3-month old homozygous mice was lower than that in the brain of 28-month old wild-type mice. Further to the phenotypes described above, myelin sheath and axon degeneration together with down-regulation of BDNF expression added to the spectrum of premature aging features in Cisd2^{-/-} mice.

A summary of the aging-related phenotypes in Cisd2^{-/-} mice was provided in Table 1. These mutant mice exhibited a premature aging phenotype with 100% penetrance for both sexes using either a C57BL/6 (B6) or a 129Sv/B6 mixed background.

TABLE 1
Aging-related phenotypes in Cisd2 knockout mice.

| Phenotype | Cisd2 knockout (−/−) mice |
|---|---|
| Median lifespan^{§} | 67 weeks (wk) |
| Minimum lifespan^{§} | 22 wk |
| Maximum lifespan^{§} | 112 wk |
| Body weight^{#} | 13% reduction at 4 wk, 27% reduc- |
| | tion at 8 wk, 41% reduction at 36 wk |
| Skeletal muscle degeneration | By 3 wk |
| Cardiac muscle | Ultrastructure (TEM) abnormality by |
| | 3 wk |
| Prominent eyes & protruding ears | By 8 wk |
| Osteopenia | By 10 wk |
| Lordokyphosis | By 12 wk |
| Abnormal pulmonary functions | By 20 wk |
| Opacity of the cornea | By 20 wk |
| Cataract formation | Not observed |
| Skin atrophy | By 48 wk |
| Hair graying | By 48 wk |
| Hair re-growth | 25% reduction at 52 wk |
| Wound healing | Normal at 52 wk |
| Cellular senescence* | No overt phenotype for MEF |
| Adipose tissue reduction^{$} | 50% reduction at 52 wk, 80% reduc- |
| | tion at 80 wk |
| Ovarian dysfunction | Histological normal at 24 wk |

^{§}In the wild-type control mice, the median lifespan is 109 wk; minimum lifespan is 72 wk; maximum lifespan is 132 wk.
^{#}Data obtained from male mice
*MEF, the mouse embryonic fibroblast was established from E13.5 embryos of wild type (+/+), heterozygote (+/−), and homozygote (−/−) mice.
^{$}Data collected from the adipose layer of cutaneous tissue.

### Example 8

### Mitochondrial Degeneration and Autophagy

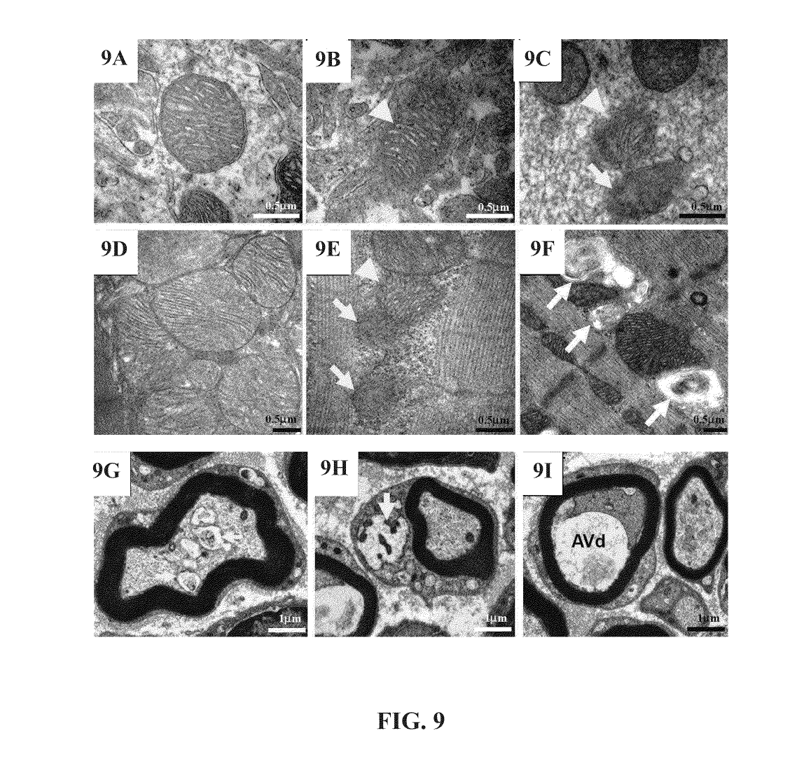

The observation of premature aging phenotypes involving muscle degeneration prompted a detailed examination of the tissue ultrastructure of the homozygous knockout mice. A TEM study revealed that mitochondrial degeneration occurred in the axons of sciatic nerves, brain cells (FIG. 9A-C), cardiac muscle cells and skeletal muscle cells (FIG. 9D-F) in the Cisd2^{-/-} mice. Notably, the mitochondrial outer membrane appeared to have broken down prior to the destruction of the inner cristae (FIGS. 9B and 9E).

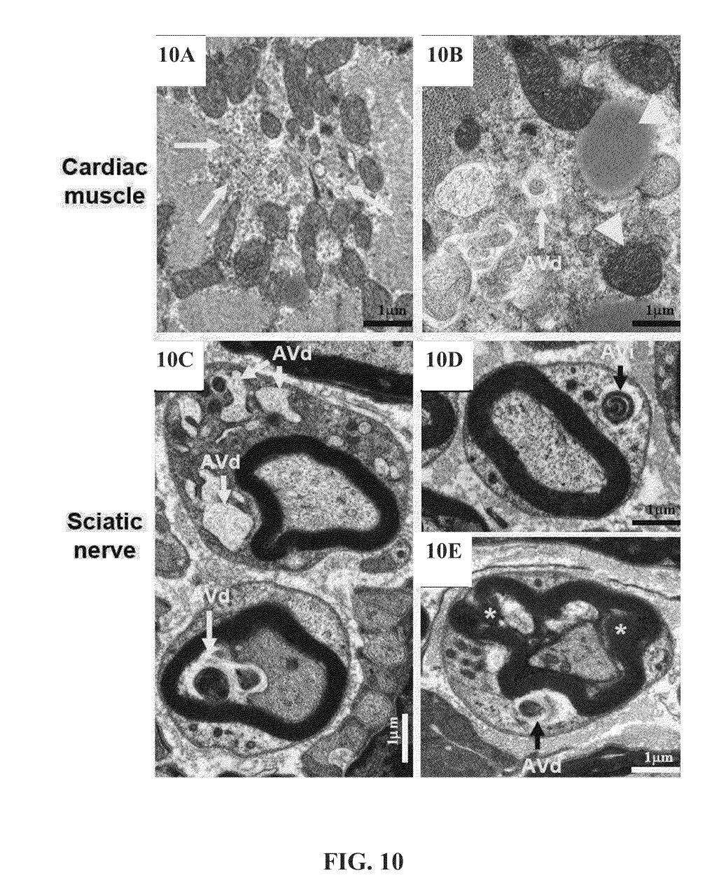

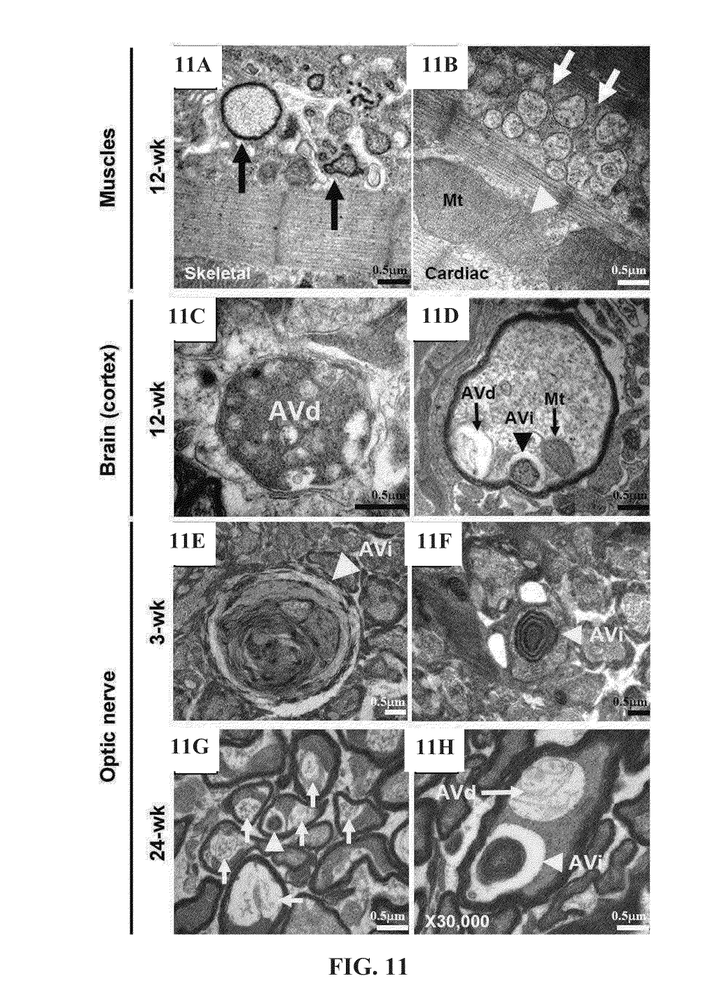

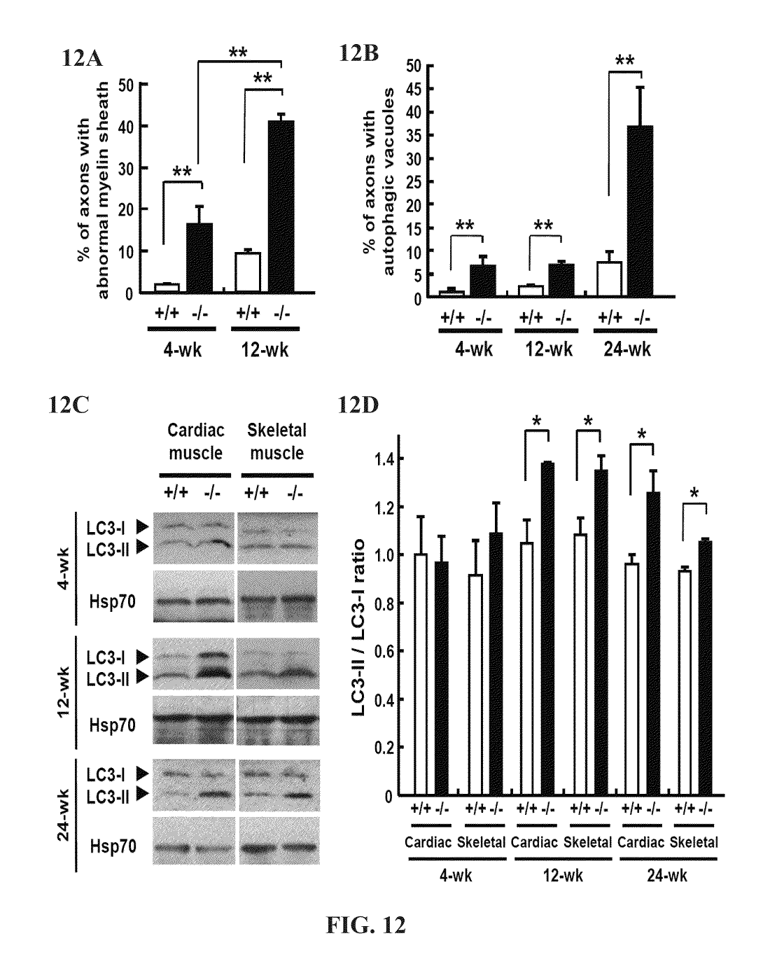

Importantly, these mitochondrial abnormalities, involving destruction of mitochondria, myelin sheath disintegration and axonal lesions, were already present to a certain extent in 2-week old Cisd2^{-/-} mice (FIG. 9G-I; FIG. 10), a stage prior to the first premature aging phenotype of muscle and nerve degeneration in these mice. Interestingly, the damaged mitochondria appeared to induce autophagy to eliminate the dysfunctional organelles because the present invention has identified morphologically distinct autophagic vacuoles in muscle, sciatic nerve, optic nerve and brain tissue (FIG. 9G-I; FIG. 11). The general term autophagic vacuole referred to an autophagosome, amphisome or autolysosome. Morphologically, autophagic vacuoles could be classified into two categories: 1) early or initial autophagic vacuoles (AVis), i.e. autophagosomes, which were double-membraned structures containing undigested cytoplasmic material or organelles; 2) late or degradative autophagic vacuoles (AVds), including amphisomes and autolysosomes, which contained partially degraded cytoplasmic material. Remarkably, mitochondrial degeneration exacerbates with age and the magnitude of the autophagy increases in parallel to the development of premature aging phenotype (FIGS. 12A and 12B). The present invention also examined the autophagosome marker LC3-II in skeletal and cardiac muscles, which were the most sensitive tissues to in vivo autophagic degradation; indeed, the ratio of LC3-II/LC3-I was significantly higher in Cisd2^{-/-} mice than in their wild-type littermates. This biochemical evidence confirmed the TEM results and provided a quantitative basis for the autophagy induction (FIGS. 12C and 12D).

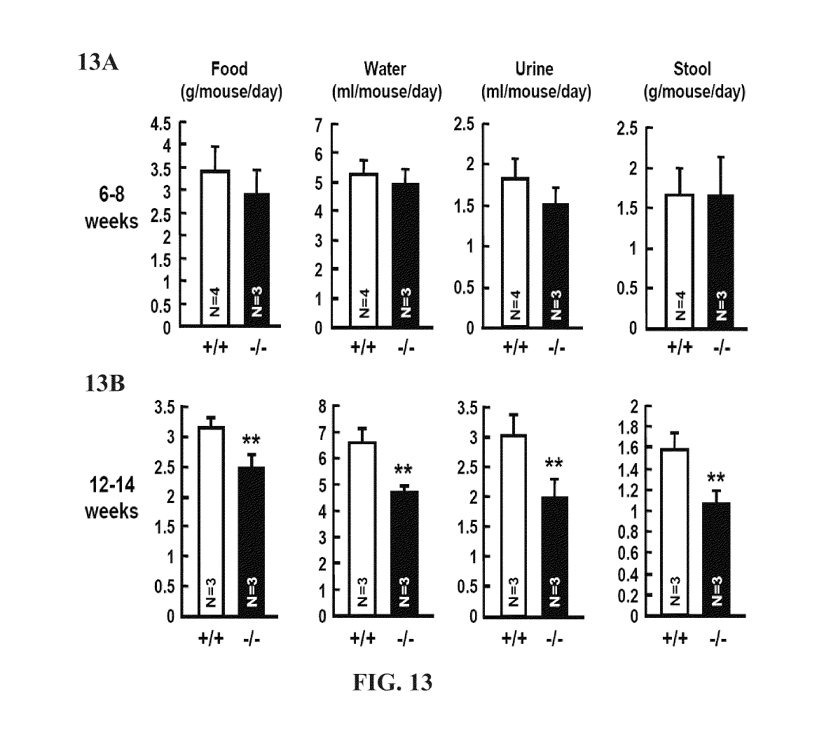

In addition, it has been reported that starvation could induce muscle autophagy. To test this possibility, the present invention measured the metabolic indices including intake of food and water and generation of urine and stool. The results of the present invention revealed no significant difference in these metabolic indices between Cisd2^{-/-} and wild-type mice at 6-week old (FIG. 13A); this was 4 weeks after the detection of autophagic activation at 2-week old. This excluded starvation/malnutrition as the cause of autophagic induction in Cisd2^{-/-} mice. A decrease in the metabolic index became evident after 12-week old (FIG. 13B) and this was likely to be a consequence of the aging phenotype.

### Example 9

### Cisd2 is a Mitochondrial Outer Membrane Protein

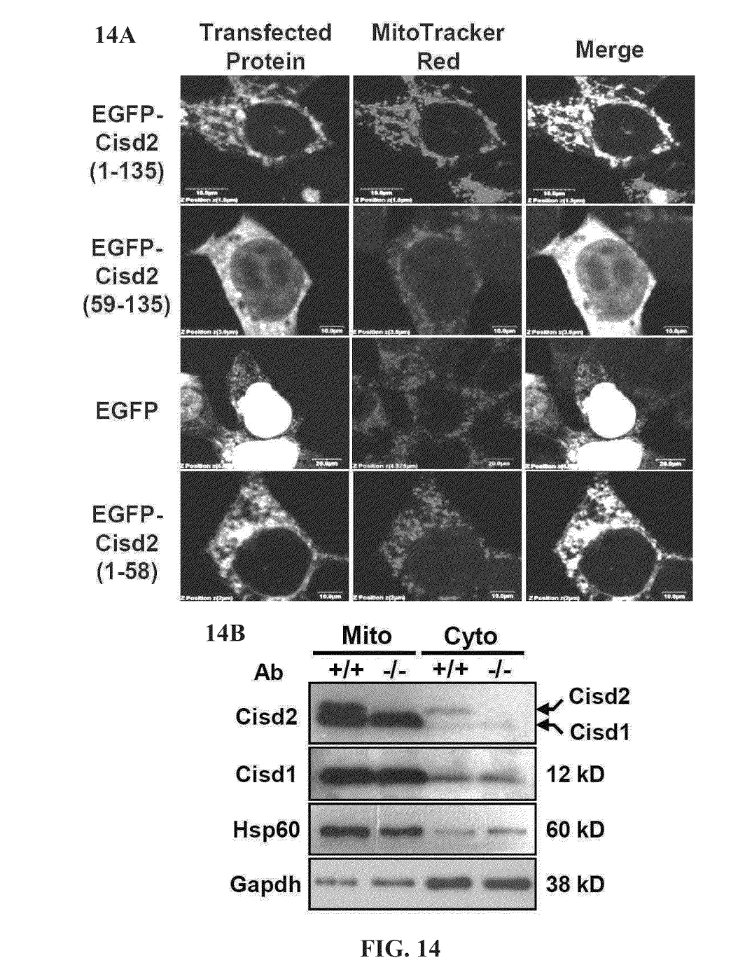

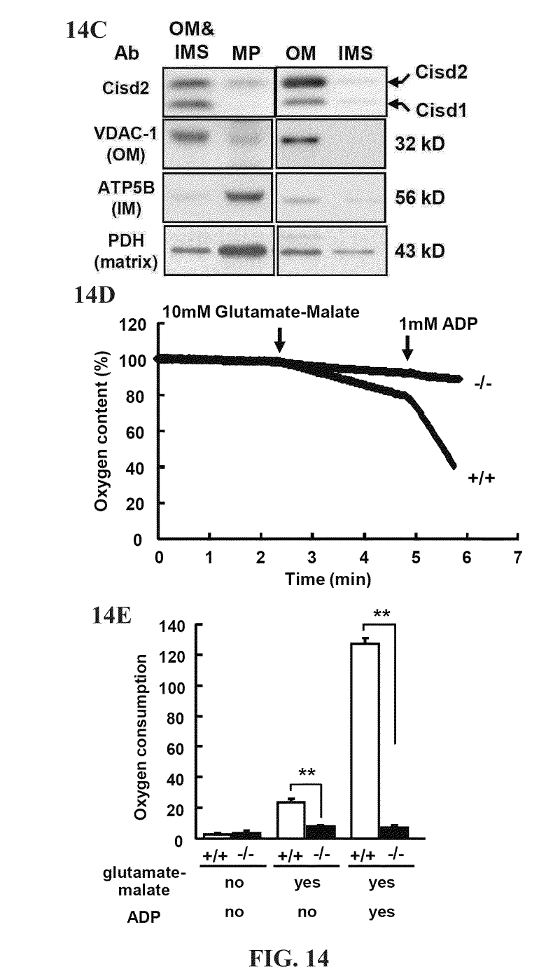

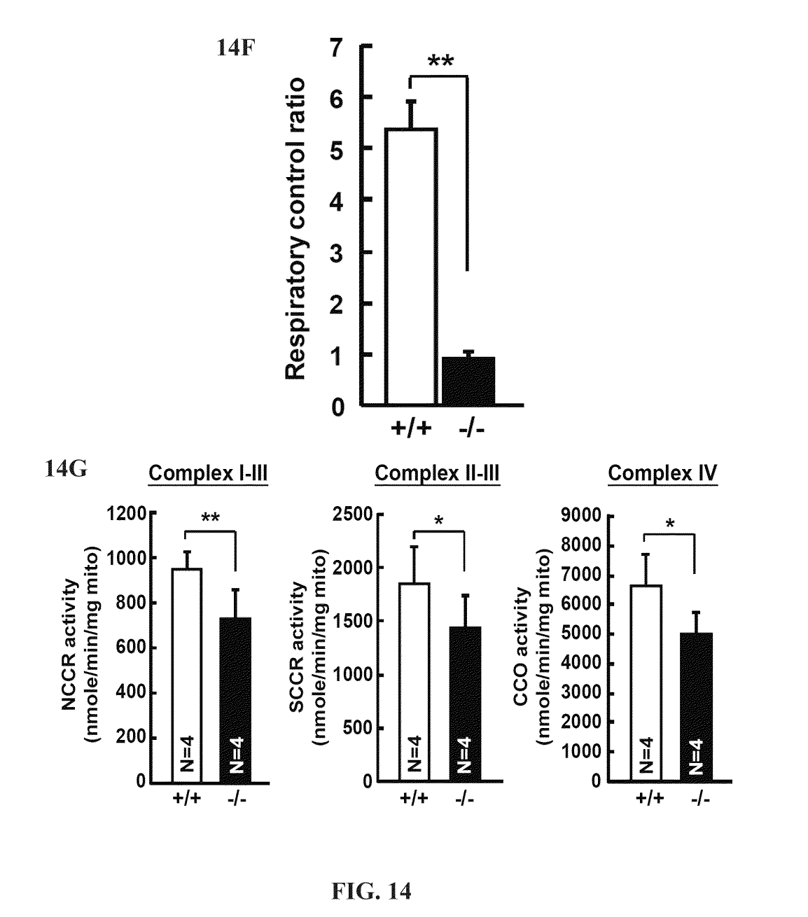

The annotated characteristics of Cisd2 protein were very similar to Cisd1, which is an outer mitochondrial membrane protein (FIG. 13A). To address the subcellular localization, the present invention expressed the EGFP-tagged Cisd2 protein in NIH3T3 cells. The result of the present invention indicated that Cisd2 was co-localized with the mitochondrial marker (FIG. 13B). However, deletion of the N-terminal 58 amino acids completely abolished the mitochondrial localization; furthermore, when the N-terminal 58 amino acids was fused to EGFP, this construct was able to redirect EGFP from a nuclear and cytoplasmic localization to the mitochondria (FIG. 14A), suggesting that Cisd2 is a nucleus-encoded mitochondrial protein and its N-terminal 58 amino acids are both necessary and sufficient to direct mitochondrial localization. To confirm the subcellular localization of the Cisd2 protein, the cytosolic and mitochondrial fractions were prepared from skeletal muscle of wild-type mice. Antibodies against Cisd1 and Cisd2 were generated. Western blot analysis revealed that Cisd2 protein, like the mitochondrial proteins Cisd1 and Hsp60, was primarily localized in the mitochondrial fraction (FIG. 14B). To further define the submitochondrial localization of Cisd2, the present invention separated mouse liver mitochondria into the following fractions: outer membrane (OM), mitoplasts (MP, inner membrane and matrix), and intermembrane space (IMS, soluble material between the inner and outer membranes) Immunoblotting each fraction with antibodies against Cisd2 and known markers revealed that Cisd2 was highly enriched in the OM fraction, as was the OM marker VDAC-1; this result strongly suggested Cisd2 is a mitochondrial outer membrane protein (FIG. 14C).

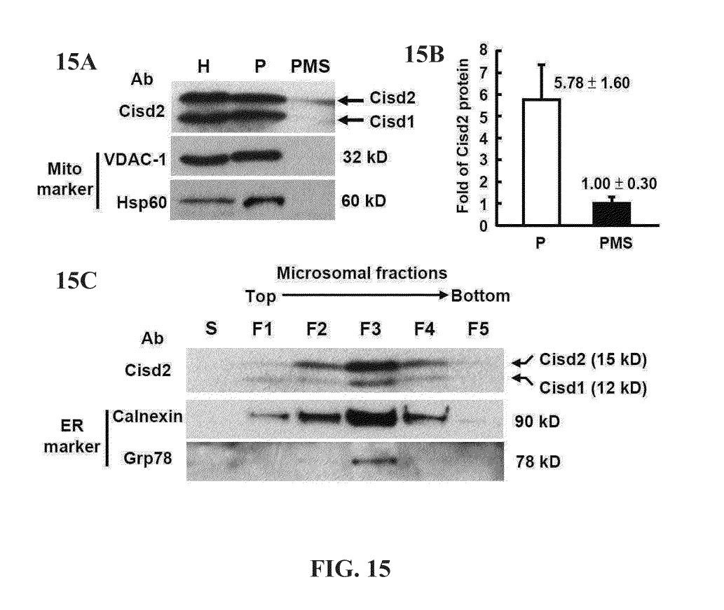

Previously a report showed that the FLAG-tagged CISD2 protein colocalized with the ER marker calnexin in the transfected mouse P19 and human HEK293 cells. The present invention sought to determine if there was a small portion of the Cisd2 protein sorted into the endoplasmic reticulum (ER)/sarcoplasmic reticulum (SR) using subcellular fractions prepared from skeletal muscles of 11 wild-type mice. The data of the present invention indeed revealed a weak signal indicating the presence of Cisd2 protein in the post-mitochondrial supernatant and this colocalized with the ER markers in the microsomal fractions. The ratio of the Cisd2 protein present in the mitochondria versus ER was estimated to be about 5.8:1 (FIG. 15).

Mitochondria are the cellular energy factories that generate ATP via oxidative phosphorylation. To investigate whether the mitochondrial degeneration detected in this study has a direct functional consequence leading to a respiratory dysfunction, the present invention assessed mitochondrial aerobic respiration using isolated mitochondria prepared from skeletal muscle. This was done by measuring the oxygen consumption after stimulating the mitochondria with glutamate-malate and ADP to activate the respiratory chain reactions. The results of the present invention revealed a significant decrease in the oxygen consumption and the respiratory control ratio in the Cisd2^{-/-} mitochondria (FIG. 14D-F). To further expand this investigation, the present invention explored the iron-sulfur proteins, which are essential electron carriers in the mitochondrial respiratory chain; there are up to 12 different iron-sulfur clusters that shuttle electrons through complex I-III. The present invention has measured the activities of the various iron-sulfur proteins of complex I-III (NADH cytochrome c reductase, NCCR) and complex II-III (succinate cytochrome c reductase, SCCR). In addition, the present invention also has measured the activity of complex IV (cytochrome c oxidase, CCO), which contains hemes and copper centers for electron transport. The results of the present invention showed that there was an average 30% decrease in the electron transport activities of complex I-III, complex II-III and complex IV in the Cisd2^{-/-} mitochondria compared with wild-type mitochondria (FIG. 14G). Together with the oxygen consumption experiment, these results revealed a respiratory dysfunction in the Cisd2^{-/-} mitochondria.

### Example 10

### WFS2 and Cisd2^{-/-} Mice

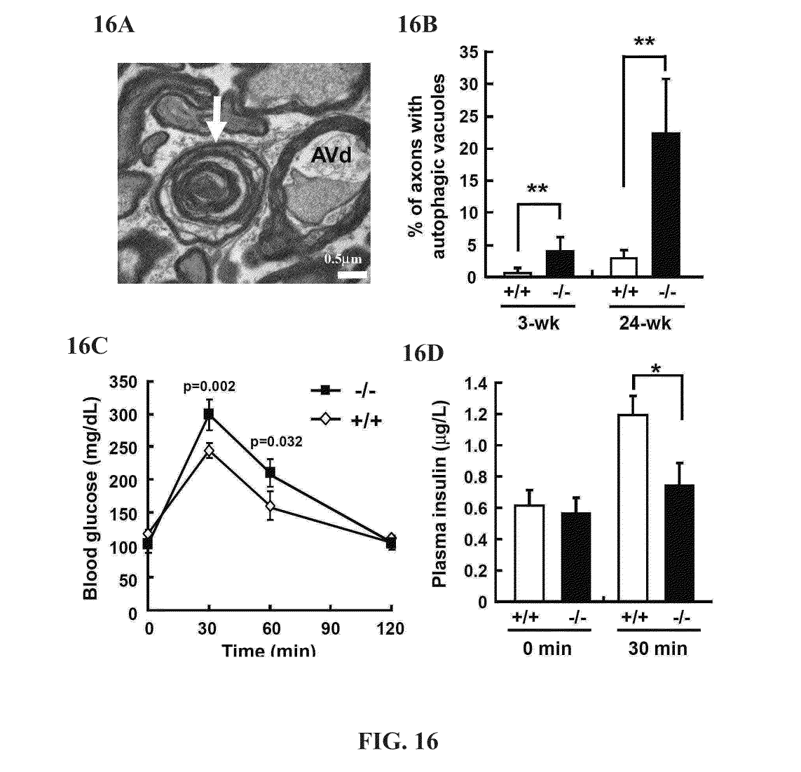

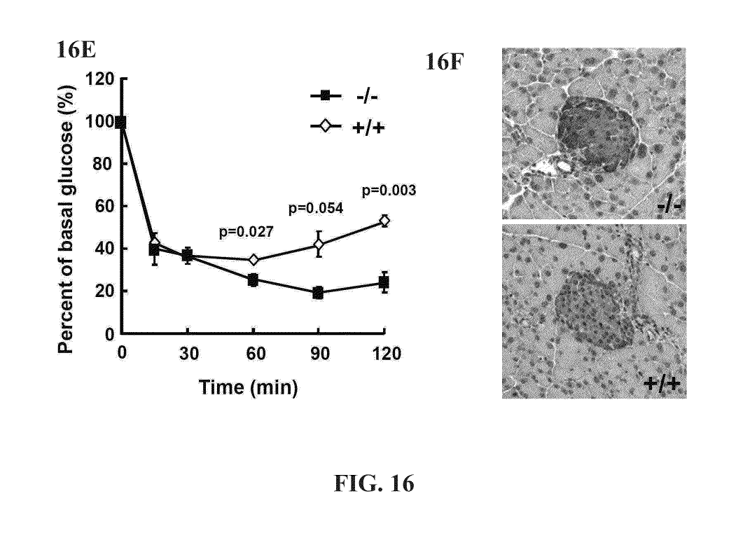

In order to evaluate the usefulness of Cisd2^{-/-} mice as an animal model for WFS2 and gain insight into the mechanistic basis of WFS2 pathogenesis, the present invention compared the clinical manifestations of this disease and the phenotype of Cisd2^{-/-} mice. WFS2 is a clinically heterogeneous disease; only juvenile-onset diabetes mellitus and optic atrophy are necessary criteria for WFS2 diagnosis. Importantly, Cisd2^{-/-} mice exhibited a progressive neurodegenerative phenotype that included optic nerve defects (FIGS. 16A and 16B; FIG. 11). Regarding glucose homeostasis, the present invention found that Cisd2^{-/-} mice display a milder phenotype, namely impaired glucose tolerance and decreased insulin secretion, which was revealed by the oral glucose tolerance test (FIGS. 16C and 16D). In addition, insulin tolerance tests did not show insulin resistant in the Cisd2^{-/-} mice; in fact, these mutant mice were somewhat more sensitive to insulin (FIG. 16E). Furthermore, IHC staining of the pancreatic islets revealed no obvious difference in insulin expression within the beta-cells between Cisd2^{-/-} and wild-type mice (FIG. 16F). Taken together, these results indicated impaired glucose homeostasis in the Cisd2^{-/-} mice, which seemed to have an insulin secretory defect rather than insulin resistance. The importance of mitochondrial dysfunction in beta-cell insulin secretion defects has been previously confirmed in other mouse models, which demonstrated that mitochondrial ATP production is a critical part of the beta-cell signaling system and allows insulin release. However, there was no overt diabetes observed in the Cisd2^{-/-} mice with the C57BL/6 congenic background. This was consistent with a previous observation that C57BL/6 background conferred a more diabetes-resistant phenotype; a similar finding of a genetic background effect also had been reported for WFS1 (wolframin) knockout mice. In addition to optic atrophy and glucose intolerance, the phenotypic features of Cisd2^{-/-} mice reflect other aspects of the clinical manifestations of WFS2 patients including early (juvenile) onset and premature death (Table 2). Thus, this mutant mouse might also provide an animal model for mechanistic investigation of Cisd2 protein function and helped with the pathophysiological understanding of WFS2.

TABLE 2
Comparison of Wolfram syndrome and Cisd2 knockout mice.

| Wolfram syndrome^{#} | | | Cisd2^{-/-} mice | | |
|---|---|---|---|---|---|
| Clinical | Median age | Frequency | | Age | |
| features | (age range) | (at 30 years) | Analysis | (week) | Phenotype |
| Juvenile onset | <20 years | — | — | <3 | Early (Juvenile) onset |
| Premature | 30 years | — | Survival curve | | Premature death |
| death | (25-49 | | | | |
| | years) | | | | |
| Diabetes | 6 years | 100% | Medi-Test Glucose: | 12 | Impaired glucose |
| mellitus | (3 weeks- | | urine strips (Macherey- | | tolerance; no overt |
| | 16 years) | | Nagel); oral glucose | | diabetes |
| | | | tolerance test | | |
| Optic atrophy | 11 years | 100% | TEM examination | 3-24 | Progressive |
| & optic nerve | (6 weeks- | | | | degeneration of optic |
| degeneration | 19 years) | | | | nerve |
| Deafness | 16 years | 67% | Acoustic startle test | 12 | Negative |
| | (5-39 | | (a sudden loud noise) | | |
| | years) | | | | |
| Diabetes | 14 years | 85% | Gross observation & | 4-12 | Negative |
| insipidus | (3 months- | | autopsy | | |
| | 40 years) | | | | |
| Renal | 20 years | 56% | Autopsy; H&E kidney | 12 | Negative |
| abnormality | (10-44 | | section; multistix 10 SG | | |
| | years) | | urine strips (Bayer)* | | |
| Neurological | 30 years | 41% | TEM examination & | 2-24 | Progressive |
| features | (5-44 | | gross observation | | degeneration of |
| & Ataxia | years) | | | | peripheral (sciatic) |
| | | | | | nerve; unsteady gait |

^{#}Clinical features are based on Barrett, T. G. & Bundey, S. E. Wolfram (DIDMOAD) syndrome. 1997. J. Med. Genet. 34, 838-841.
*Multistix urine strips included testing for: Bilirubin, blood, glucose, ketones, leukocytes, nitrite, pH levels, protein, specific gravity, and urobilinogen.

### Example 11

### The study of Resveratrol (RES) in Cisd2^{-/-} Mice

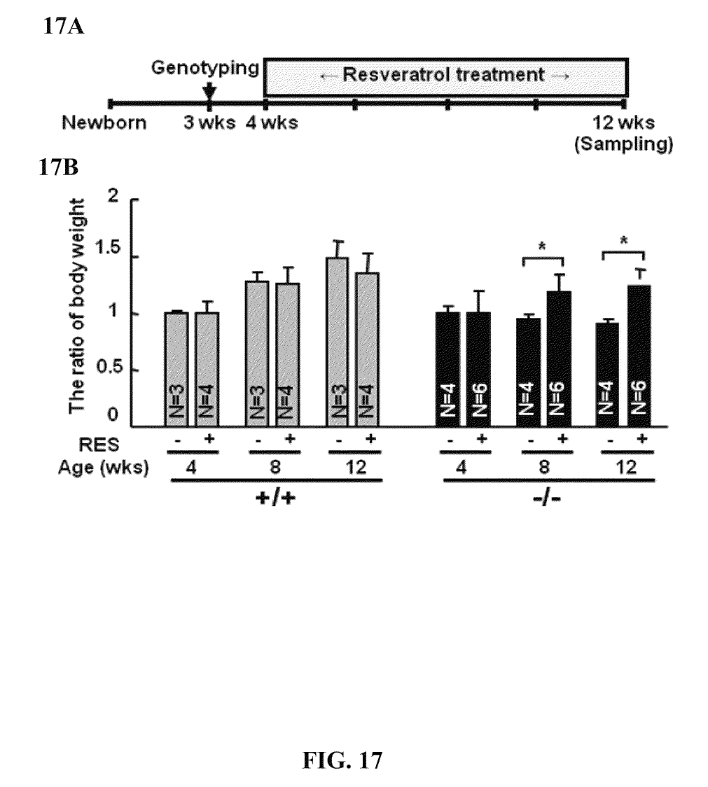

Resveratrol (30 mg/kg/day) was administered by oral to Cisd2 knockout mice from 4- to 12-week old. The body weight of the Cisd2 knockout mice was analyzed after resveratrol treatment and comparing to 4-week old wild-type mice (FIG. 17). Data showed that the body weight of Cisd2 knockout mice has significant reverse after 8-week old.

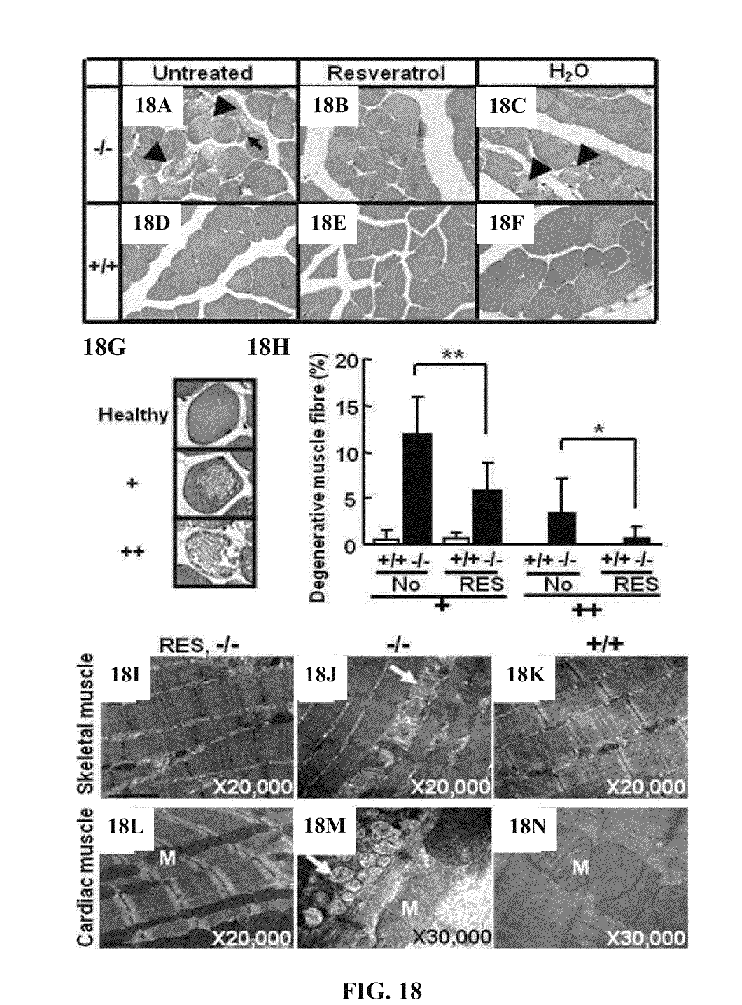

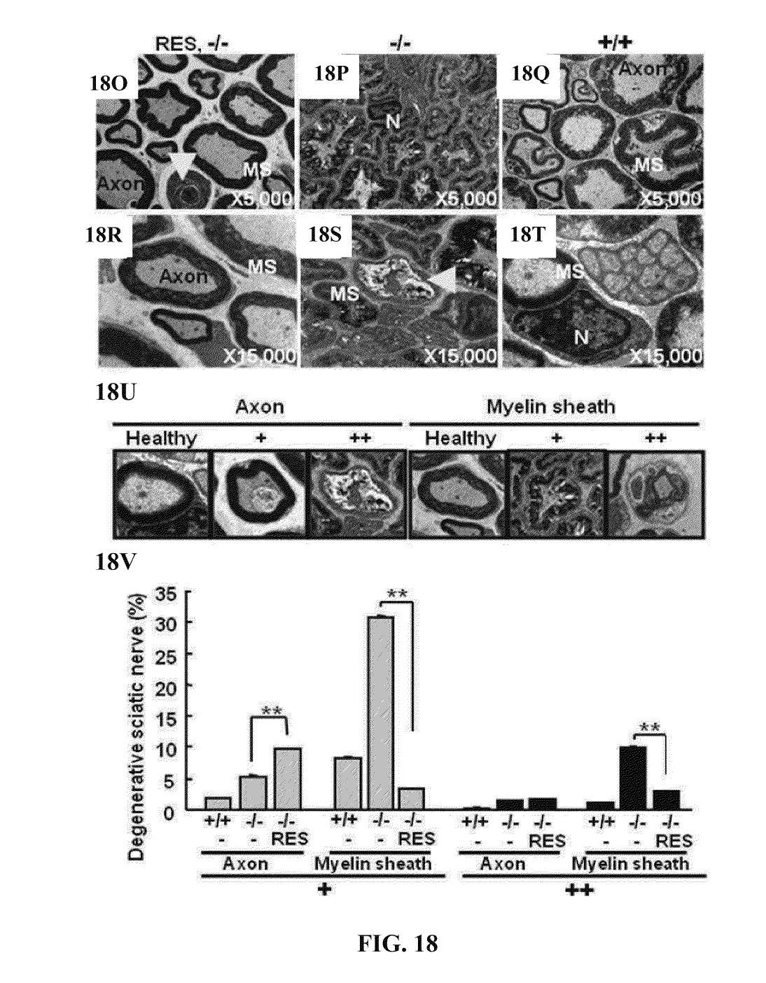

Results in FIG. 18 showed that resveratrol treatment has partial rescue on muscle and neuron degeneration of the Cisd2 knockout mice. H&E staining of transverse sections of skeletal muscle dissected from the 12-week old, male Cisd2 knockout mice with untreated control, resveratrol or H₂O treatment, and relative wild-type control. The standard quantification score of muscle atrophy was prepared. The quantification of degenerative muscle fiberin was measured under resveratrol treated or not. About 1000 muscle fibres in random fields of H&E staining slides were examined for each mouse. Ultrastructure of skeletal and cardiac muscle was dissected from Cisd2 knockout mice with resveratrol treatment, Cisd2 knockout mice without resveratrol treatment, and 12-week old wild-type, respectively. Ultrastructure of a myelinated nerve fiber was dissected from the sciatic nerve of Cisd2 knockout mice with resveratrol treatment, Cisd2 knockout mice without resveratrol treatment, and 12-week old wild-type, respectively. The axon is enveloped with myelin sheath (MS) formed by fusion of many layers of Schwann cell plasma membrane. Myelin sheath degeneration was detected only in the Cisd2 knockout mice. The standard quantification score of axon and myelin sheath degeneration was created. The quantification of degenerative sciatic nerve was shown in wild-type, Cisd2 knockout mice without resveratrol treatment and Cisd2 knockout mice with resveratrol treatment, respectively. About 500 axons in random fields of TEM's grids were examined for each mouse.

Insofar as the description above and the accompanying drawing disclose any additional subject matter that is not within the scope of the single claim below, the inventions are not dedicated to the public and the right to file one or more applications to claim additional inventions is reserved.

Although a very narrow claim is presented herein, it should be recognized the scope of this invention is much broader than presented by the claim. It is intended that broader claims will be submitted in an application that claims the benefit of priority from this application.

What is claimed is:

1. A transgenic knock-out mouse whose genome comprises a homozygous disruption in its endogenous CDGSH iron sulfur domain 2 (CISD2) gene, wherein said CISD2 is defined as SEQ ID NO. 1, and wherein said homozygous disruption results in said transgenic knockout mouse exhibiting decreased levels of CISD2 as compared to a wild-type mouse, said mouse showing symptoms similar to the features of premature aging, said features consisting of optic atrophy, neurological features, ataxia, cardiac and skeletal muscle degeneration and nerve demyelination and neuron degeneration.

2. The transgenic knock-out mouse of claim 1, wherein the optic atrophy is a phenotype of progressive degeneration of optic nerve starts from age 3-week of the transgenic knock-out mouse.

3. The transgenic knock-out mouse of claim 1, wherein the ataxia is a phenotype of progressive degeneration of sciatic nerve and unsteady gait starts from age 2-week of the transgenic knock-out mouse.

4. The transgenic knock-out mouse of claim 1, wherein the feature of cardiac and skeletal muscle degeneration starts from age 3-week of the transgenic knock-out mouse.

5. The transgenic knock-out mouse of claim 1, wherein the feature of nerve demyelination and neuron degeneration starts from age 3-week of the transgenic knock-out mouse.


SEQUENCE LISTING (5 sequences)

SEQ ID NO: 1 - DNA, 2881 bp, Mus musculus
  gene (1)..(2881)
ccgcgagcccgccggccgcttccgctctcacgcaagcgcagcagccgggcagcggacgccgccggcggcaggatggtcctggacagcgtggcccgcatcgtgaaggtgcagctgcccgcctacctcaagcagctcccggtccccgacagcatcaccgggttcgcccgcctcacagtttcagactggctccgcctactgcccttcctcggggtacttgcgcttctgggctacctcgcagtgcgcccattcttcccaaagaagaagcaacagaaggatagcttgatcaatcttaagatacaaaaggaaaatcccaaggtggtgaatgagataaacattgaagatctgtgtctcaccaaagcagcttattgtaggtgctggcggtccaagacgtttcctgcctgtgatggatcccataataagcataatgaattgacaggcgataacgtgggtcctctcatcctgaagaagaaagaagtatagcagtggccatttgagatgaaactctgacagagtgtttccatgtgtgttgtgcatagaaaagtctttggaagttttaactctcagtactggttgaataattatttctgccagttattttcttgctacactactgcttatatttggtactttatatagtcagtcatttctatagaaatcagatggtcaagtctcattctgaaattaagtttaatgtgtctttcaacaataaagtcagcacttcagattttaattaattgggaatttaaagtttgactgtgggccaaaagttatttctttgtatatcagtatttcaacagaatacatagttttaaaaaaattatcctttgttctgttaaatgtatctttatattgtcttgttttgctcatttgaagtcatttgccatttttttggccttttgtggaactctgctgcttttagaagaattatgaaaaataattgccatcaacaatattttgaggcagtaaaaccccttgttttatagagactttcatcttattaccatgattatcccaatttctaaaaagtaaatcagatataaaaatttccaattagaaacaattattttcatcataaaaatggttcaccttacaaaagccttgttgtgtgtgtgagggtgtgagggtgtatatgtgtgtgtgagtatgtgtgtgtgtgtgagagagagagagtatgtgtgtgtgtgtgtggtgcttaacttggagactgctgggtggcaagagcactcatgaactagagtttgaagatcagttagatattaaataactgaatttgcattaaaatgatgcatagttcattgctcaaaattatgttttatcttttctgttgtaaattatagagaaacggcaacaggatattaagttcttgtattctaatggattgaatttgtacatactgagagaaaatattttgatcttttttaaaagagataactcttgttgtatttttttaacgtcacaaaatatagatcaaacacttgtgggcataatgacagaaaaaaagtctggaaggaccaaggactatgactatgacacagcatggtccacttgcacggataaggacgaggacttagcaaaggttgctgcttttactctctatttctcttccccccccccccccccgcaacaaacctttatatgctttgcttttaattgatcttttcttaattgaggctttgaatagaatcgtcatagcacagttaagctctttaggcccgagtgataagacctgattgattgtctaaggatagggtggagccagcagggcaggaagcatcatcacaagtggattagacagaggctgttcgaggagaggaagccacacacgtgttacagcaaatactgaaaaaaccaaaacaccaacagtcctttcacgtctggctcctaagtgaaatgacacgtcaagggcaaggtaggctaaataaagaaggtgggctttaagtatgtacttcagttctgccgtgttcccccagaagcagggtaaaagcaagacagttaaatctggggacactcacctgtctgcctgctggagtagatggaccatctctaggatttcttctttattaaatgagcacacagtatgcctggtaccacatgaattctgtttgttctctttctttagctatcctatctagcagtttgagaaagactagtgatggcaagctaaaaaggaaaatgattaccagtcaacttgtcacaaaattaagttacactgacacagtttgtagtcttagtttgtgtattgtaaagtgtaaataatgttaaaacagacagacatgttttagaaagacttatttcatgtgtatgagtgcctgtctgcctgtctgtgtgtctgtctgtgcaccatgcacatgcctggtgcccgaggaagccagagagaatgttggatccctggaactggaattaacagatagttgtgagctaccatgtgaatactgggaactgaacctggtctcctacaagaacatccagtgctattaacaactgagccatctctctaaccctataaaacttttaataaaagaggttatgattggtttgaagtagcaactgaaatgtggaaggaagtattccgaaaggcagaagtggtacaaagccgtgtctgctcactccactcatggtactgaggatggaacccagggcctcccacaccctggcaagagcccttcacactgagctccgtcttgagttgtatcttttcaatagcataaaccattgagacaaaatacaagaaagttttccatataggtttactggaatttttgaattatttgtgattttagcctactatcagaagtctctttagttttattttacaagtctcatgtaagagctattcttaaactattaaagatacaacctactcgttaaaaaaaaaaaaaaa

SEQ ID NO: 2 - DNA, 19 bp, Artificial
  primer
acttgcgcttctgggctac

SEQ ID NO: 3 - DNA, 20 bp, Artificial
  primer
ccagcacctacaataagctg

SEQ ID NO: 4 - DNA, 21 bp, Artificial
  primer
gaattgacaggcgataacgtg

SEQ ID NO: 5 - DNA, 21 bp, Artificial
  primer
tataagcagtagtgtagcaag